(19) United States
(12) Patent Application Publication
(10) Pub. No.: US 2014/0323497 A1
(43) Pub. Date: Oct. 30, 2014

(54) METHODS AND FORMULATIONS OF TREATING THROMBOSIS WITH BETRIXABAN AND A P-GLYCOPROTEIN INHIBITOR

(71) Applicant: Portola Pharmaceuticals, Inc., S. San Francisco, CA (US)

(72) Inventors: Uma Sinha, San Francisco, CA (US); George A. Mansoor, Rahway, NJ (US); Anne Hermanowski Vosatka, Rahway, NJ (US); Vincent R. Capodanno, Rahway, NJ (US); Robert M. Wenslow, Rahway, NJ (US); Richard G. Ball, Rahway, NJ (US); Eric L. Margeiefsky, Rahway, NJ (US); Timothy K. Maher, Rahway, NJ (US); Itzia Zoraida Arroyo, Rahway, NJ (US); Brett Lauring, Rahway, NJ (US); Liam Corcoran, Tipperary (IE); William Denney, Lansdale, PA (US); Michael McNevin, Summit, NJ (US); Anjali Pandey, Fremont, CA (US)

(73) Assignee: Portola Pharmaceuticals, Inc., S. San Francisco, CA (US)

(21) Appl. No.: 14/078,415

(22) Filed: Nov. 12, 2013

Related U.S. Application Data
(63) Continuation of application No. 13/223,038, filed on Aug. 31, 2011, now abandoned.
(60) Provisional application No. 61/379,339, filed on Sep. 1, 2010.
(60) Provisional application No. 61/454,402, filed on Mar. 18, 2011.

(51) Int. Cl. A61K 31/44; A61K 31/343; A61K 31/496; A61K 31/277

(52) U.S. Cl. CPC A61K 31/44; A61K 31/277; A61K 31/343; A61K 31/496
USPC 514/254.07; 514/353

(57) ABSTRACT

This invention is directed to methods of inhibiting coagulation or treating thrombosis using a factor Xa inhibitor and a P-glycoprotein (Pgp) inhibitor. The invention is also directed to formulations used in the methods.

### CROSS-REFERENCE TO RELATED APPLICATIONS

This application claims the benefit under 35 U.S.C. §119(e) of U.S. Provisional Application Nos. 61/379,339, filed on Sep. 1, 2010, and 61/454,402, filed on Mar. 18, 2011, the contents of each of which are hereby incorporated by reference in their entirety into the present disclosure.

### BACKGROUND OF THE INVENTION

1. Field of the Invention

This invention is directed to methods of inhibiting coagulation or treating thrombosis using a factor Xa inhibitor, such as betrixaban, and a P-glycoprotein (Pgp) inhibitor. The invention is also directed to formulations used in the methods.

2. State of the Art

Factor Xa is a serine protease, the activated form of its precursor factor X, and a member of the calcium ion binding, gamma carboxyglutamic acid (GLA)-containing, vitamin K dependent, blood coagulation factors. Factor Xa appears to have a single physiologic substrate, namely prothrombin. Since one molecule of factor Xa may be able to generate greater than 1000 molecules of thrombin (Mann, et al., J. Thrombosis. Haemostasis 1: 1504-1514, 2003), direct inhibition of factor Xa as a way of indirectly inhibiting the formation of thrombin has been considered an efficient anticoagulant strategy.

Several classes of small molecule factor Xa inhibitors have been reported, for example, in U.S. Pat. Nos. 6,376,515, 7,521,470, and 7,696,352, U.S. Patent Application Publication Nos. 2007/0259924, 2008/0293704, and 2008/0051578, all of which are incorporated by reference in their entirety.

U.S. Pat. Nos. 6,376,515 B2 and 6,835,739 B2, the contents of which are incorporated herein by reference, disclose a specific factor Xa inhibitor compound, [2-({4-[(dimethylamino)iminomethyl]phenyl}carbonylamino)-5-methoxyphenyl]-N-(5-chloro(2-pyridyl))carboxamide (betrixaban), which has the following structure:

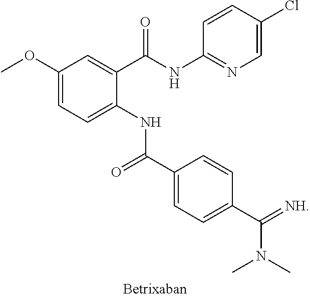 [SMILES: COc1ccc(NC(=O)c2ccc(C(=N)N(C)C)cc2)c(C(=O)Nc2ccc(Cl)cn2)c1]

Since treatment for diseases such as acute coronary syndromes might require co-administration of an anticoagulant agent and an antiplatelet agent, a combination would allow for increased efficacy as well as superior patient compliance during chronic treatment. However, some current anticoagulant therapies are not suitable for combination therapy. For example, warfarin, a currently available anticoagulant for chronic use, requires dose titration using international normalized ratio (INR) clotting assays to avoid excessive blood thinning and the risk of bleeding. Therefore, it cannot be used as a combination with an antiplatelet agent at a fixed dose. Further, while some anticoagulant agents and antiplatelet agents may be suitable for combination therapy, they do not provide sufficient therapeutic benefit. For example, a recent clinical study examined patients on fondaparinux (an anticoagulant agent) and either aspirin or clopidogrel. The patients continued to experience thrombotic events over the course of the study. (Fifth Organization to Assess Strategies in Acute Ischemic Syndromes Investigators, et al, N. Engl. J. Med. 2006, 354(14):1464-76).

### SUMMARY OF THE INVENTION

It is herein discovered that concomitant administration of a P-glycoprotein (Pgp) inhibitor significantly increases the exposure of a factor Xa inhibitor in the patient. Accordingly, to achieve the same therapeutic objective of the factor Xa inhibitor when administered alone, a lesser amount is required when co-administered with a Pgp inhibitor.

In particular, it is demonstrated herein that the plasma concentration of betrixaban, a factor Xa inhibitor, was increased when co-administered with any of the Pgp inhibitors: ketoconazole, amiodarone and verapamil. By contrast, co-administration with digoxin, a Pgp substrate not inhibiting the activity of Pgp, did not alter the exposure of betrixaban significantly.

It is surprising, however, the increase of betrixaban by ketoconazole, amiodarone and verapamil was about 2.2-2.4 folds, 2.5-2.7 folds and 2.9-4.7 folds, respectively, whereas ketoconazole is known to be a stronger Pgp inhibitor than amiodarone which, in turn, is known to be a stronger Pgp inhibitor than verapamil. It is further contemplated, therefore, that the synergistic effect between betrixaban and Pgp inhibitors is also impacted by the dosing schedule. In this respect, concurrent administration may lead to higher synergism than separate administration.

It is further contemplated that co-administration with betrixaban, either at a therapeutic dose or subtherapeutic dose, increases the exposure of these Pgp inhibitors. In the same vein, such co-administration reduces the amount of the Pgp inhibitors required to achieve a therapeutic objective, thereby reducing potential side effects. In some embodiments, the Pgp inhibitor is selected from verapamil, amiodarone or ketoconazole.

Thus, in one embodiment, the present disclosure provides a method for treating thrombosis or inhibiting blood coagulation in a patient receiving administration of a P-glycoprotein inhibitor, the method comprising administering to the patient a subtherapeutic dose of betrixaban.

In one embodiment, the amount of betrixaban administered is about 20% less than the therapeutically effective amount. In one embodiment, the amount of betrixaban administered is about 50% less than the therapeutically effective amount. Alternatively, the amount of betrixaban administered is about 25%, 30%, 35%, 40%, 45%, 50%, 55%, 60%, 65%, 70%, 75%, 80%, 85% or 90% less than the therapeutically effective amount.

A therapeutically effective amount of betrixaban, depending on the patient and/or condition, such as body weight, of the patient, can be about 40 mg, 60 mg, 80 mg, 90 mg, 110 mg, 130 mg, or 150 mg aggregate daily dose. In some aspects, the aggregate daily dose is further adjusted based on the body weight and/or gender of the patient. In a particular aspect, the aggregate daily betrixaban dose for a human patient is about 40 mg. In another aspect, the aggregate daily betrixaban dose for a human patient is about 60 mg. In yet another aspect, the aggregate daily betrixaban dose for a human patient is about 80 mg.

Accordingly, in any one of the above embodiments, the amount of betrixaban administered is from about 25 to about 35 mg. In another embodiment, the amount of betrixaban administered is from about 20 to about 35 mg. In another embodiment, the amount of betrixaban administered is from about 15 to about 35 mg. In another embodiment, the amount of betrixaban administered is from about 10 to about 35 mg. In another embodiment, the amount of betrixaban administered is from about 25 to about 30 mg. In another embodiment, the amount of betrixaban administered is from about 15 to about 30 mg. In another embodiment, the amount of betrixaban administered is from about 10 to about 30 mg. In another embodiment, the amount of betrixaban administered is from about 15 to about 20 mg. In another embodiment, the amount of betrixaban administered is from about 10 to about 20 mg. In another embodiment, the amount of betrixaban administered is from about 10 to about 15 mg.

In one embodiment, the patient receives the administration of the P-glycoprotein inhibitor at least half an hour before or after administration of betrixaban. In another embodiment, the patient is concurrently administered with the P-glycoprotein inhibitor and betrixaban.

In any of the above embodiment, the patient receives administration of an therapeutically effective amount of the P-glycoprotein inhibitor, or alternatively a subtherapeutic dose of the P-glycoprotein inhibitor. In some embodiments, the P-glycoprotein inhibitor is in a controlled release form.

P-glycoprotein inhibitors, without limitation, include verapamil, amiodarone and ketoconazole.

For verapamil, the exemplary dose is about 100 mg to about 300 mg. For amiodarone, the exemplary dose is about 200 mg to about 600 mg. For ketoconazole, the exemplary dose is about 100 mg to about 300 mg.

In any of the above embodiments, betrixaban is in the form of a pharmaceutically acceptable salt, such as a maleate salt. In one aspect, the maleate salt is in a crystalline form selected from the group consisting of Form I, Form II, Form III and combinations thereof.

In some embodiments, the thrombosis is associated with a condition selected from the group consisting of acute coronary syndrome, myocardial infarction, unstable angina, refractory angina, occlusive coronary thrombus occurring post-thrombolytic therapy or post-coronary angioplasty, a thrombotically mediated cerebrovascular syndrome, embolic stroke, thrombotic stroke, transient ischemic attacks, venous thrombosis, deep venous thrombosis, pulmonary embolus, coagulopathy, disseminated intravascular coagulation, thrombotic thrombocytopenic purpura, thromboangiitis obliterans, thrombotic disease associated with heparin-induced thrombocytopenia, thrombotic complications associated with extracorporeal circulation, thrombotic complications associated with instrumentation, and thrombotic complications associated with the fitting of prosthetic devices.

In some embodiments, the thrombosis is associated with a condition selected from the group consisting of thromboembolic stroke, ischemic stroke, hemorrhagic stroke, systemic embolism, stroke in atrial fibrillation, non-valvular atrial fibrillation, venous thromboembolism (VTE), myocardial infarction, deep venous thrombosis, and acute coronary syndrome (ACS).

Further, in some embodiment, the treatment of thrombosis is for stroke prevention in atrial fibrillation (SPAF), prevention of VTE in knee or hip surgery, prevention of VTE in acute medically ill patients, prevention of arterial thrombosis in acute coronary syndrome patients, secondary prevention in acute coronary syndrome, secondary prevention of myocardial infarction, stroke or other thrombotic events in patients who have had a prior event.

In a particular embodiment, the treatment of thrombosis is for stroke prevention in a patient with atrial fibrillation. In another embodiment, the patient is a patient with atrial fibrillation or atrial flutter.

Also provided is an unit dose comprising from about 10 to about 20 mg of betrixaban and an effective amount of a P-glycoprotein inhibitor. In some embodiments, the P-glycoprotein inhibitor is selected from the group consisting of verapamil, amiodarone and ketoconazole.

Further provided is a method for treating thrombosis or inhibiting blood coagulation, the method comprising administering to the patient a synergistically effective amount of betrixaban, wherein the patient is not currently under treatment with a P-glycoprotein inhibitor.

### BRIEF DESCRIPTION OF THE DRAWINGS

FIGS. 1A and 1C provide betrixaban plasma concentrations of patients administered betrixaban alone. FIGS. 1B and 1D provide betrixaban plasma concentrations of patients administered betrixaban and amiodarone. As explained further in Example 1, the co-administration of amiodarone increased the plasma concentrations of betrixaban as evident from comparing 1A and 1B, and 1C to 1D.

FIG. 2 presents the mean betrixaban plasma concentration-time profiles by treatment group for all subjects, whether treated with betrixaban alone with along with ketoconazole. Mean plasma concentrations of betrixaban were quantifiable up to 96 hours after a single oral dose of betrixaban at 40 mg.

FIG. 3 presents the mean ketoconazole plasma concentration-time profiles by treatment for all subjects, whether treated with ketoconazole alone or alone with betrixaban. Mean plasma concentrations of ketoconazole were quantifiable up to 12 hours after a dosing with 200 mg ketoconazole.

FIG. 4 shows the individual and mean Cₘₐₓ of betrixaban after single oral administration of 40 mg betrixaban alone or with ketoconazole.

FIG. 5 shows the individual and Mean AUC_{(0-∞)} of betrixaban after single oral administration of 40 mg betrixaban alone or with ketoconazole.

FIG. 6 shows the individual ratios, geometric mean ratios (GMR: betrixaban+verapamil/betrixaban alone), and the corresponding 90% confidence intervals of AUC_{0-∞}. (hr*ng/mL) for betrixaban after co-administration with verapamil on days 1 and 14 in healthy volunteers.

FIG. 7 shows the individual ratios, geometric mean ratios (GMR: betrixaban+verapamil/betrixaban alone), and the corresponding 90% confidence intervals of Cₘₐₓ (ng/mL) for betrixaban after co-administration with verapamil on days 1 and 14 in healthy volunteers.

FIG. 8 shows the mean plasma concentration profiles for betrixaban following a single 40 mg oral dose of betrixaban alone or following administration of 240 mg of verapamil HCl SR QD for 18 days with single doses of 40 mg betrixaban co-administered with verapamil on days 1 and 14 to healthy subjects (insert: semi-log scale).

### DETAILED DESCRIPTION OF THE INVENTION

Before the compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

When a numerical designation is preceded by the term “about”, it varies by (+) or (−) 10%, 5% or 1%. When “about” is used before an amount, for example, in mg, it indicates that the weight value may vary (+) or (−) 10%, 5% or 1%.

### 1. DEFINITIONS

In accordance with the present invention and as used herein, the following terms are defined with the following meanings, unless explicitly stated otherwise.

As used in the specification and claims, the singular form “a”, “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this invention.

The term “aggregate daily dose” refers to the amount of a drug or compound administered in a period of about 24 hours.

The term “controlled release”, “slow release” or “extended release” refers to a drug formulation used in pill tablets or capsules to dissolve slowly and release the drug over time. In one embodiment, the drug retains at least about 50% of Cₘₐₓ at about 1 hour after administration. In other embodiments, the drug retains at least about 20%, 30%, 40%, 50%, 60%, 70% or 80% of Cₘₐₓ at about 1 hour, or 2 hours, or 4 hours, or alternatively about 30 minutes, 20 minutes or 10 minutes after administration

As used herein, the term “condition” refers to a disease state for which the compounds, salts, compositions and methods of the present invention are being used.

As used herein, the term “patient” or “subject” refers to mammals and includes humans and non-human mammals. In one embodiments herein, the patient or subject is a human. In a particular embodiment, the patient is in need of a treatment to treating thrombosis or inhibiting coagulation.

“Treat” or “treating” or “treatment” of a disease or condition in a patient refers to 1) preventing the disease or condition from occurring in a mammal, in particular, a mammal who is predisposed or does not yet display symptoms of the disease or condition; 2) inhibiting the disease or condition or arresting its development; or 3) ameliorating or causing regression of the disease or condition.

“P-glycoprotein inhibitor” or “Pgp inhibitor” refers to a compound that inhibits activity of a P-glycoprotein. P-glycoproteins (P-gp or Pgp) are part of efflux transporters of the ATP-binding cassette (ABC) transporter subfamily. P-gp is also called ABCB1, ATP-binding cassette sub-family B member 1, MDR1, and PGY1. Examples of Pgp inhibitors include but are not limited to amiodarone, ketoconazole, clarithromycin, verapamil, diltiazem, cyclosporine, quinidine, erythromycin, itraconazole, ivermectin, mefloquine, nifedipine, ofloxacin, propafenone, ritonavir, tacrolimusvalspodar (PSC-833), zosuquidar (LY-335979), elacridar (GF120918), HM30181AK, R101933, and R102207, or a pharmaceutically acceptable salt thereof.

The term “subtherapeutic dose” when used to describe the amount of a factor Xa inhibitor or a Pgp inhibitor refers to a dose of the factor Xa inhibitor or the Pgp inhibitor that does not give the desired therapeutic effect for the disease being treated when administered alone to a patient. This can also be referred to as a “synergistically effective amount”, referring to the synergy observed when administering the compounds together.

The term “co-administration” or “concomitant administration” refers to two or more therapeutic compositions being administered to the same subject during a treatment period. In one embodiment, one of the two or more therapeutic compositions is administered before the therapeutic effect of another diminishes in the subject. In one embodiment, the two or more therapeutic compositions are administered within about 24 hours. In another embodiment, the two or more therapeutic compositions are administered within about 3 hours. In yet another embodiment, the two or more therapeutic compositions are administered within about one hour.

One particular embodiment of concomitant administration is “concurrent administration” which refers to two or more therapeutic compositions being administered to the same subject either during the same administration route or substantially at the same time. In one embodiment, the two or more therapeutic compositions are administered with about 30 minutes.

### 2. METHODS OF INHIBITING BLOOD COAGULATION

Factor Xa inhibitors are used to inhibit blood coagulation and related diseases and conditions. In vitro and in vivo experiments have demonstrated betrixaban's efficacy in inhibiting blood coagulation. Co-administration of drugs such as factor Xa inhibitors with therapeutic agents that may cause adverse effect due to drug-drug interactions, however, should be avoided.

It has been reported that a combination of a Pgp inhibitor and another therapeutic agent may cause side effects due to drug-drug interactions. For example, combinations of anti-microtubule drugs with potent Pgp modulators are found to be disruptive to the integrity of the blood-brain barrier. See, for instance, Inez C. J. et al., P-Glycoprotein Inhibition Leads to Enhanced Disruptive Effects by Anti-Microtubule Cytostatics at the In vitro Blood-Brain Barrier, Pharmaceutical Research, Vol. 18, Number 5, 587-592 (2001).

It has been surprisingly discovered that a Pgp inhibitor and a factor Xa inhibitor can be safely used in combination and also allows the factor Xa inhibitor, for example, betrixaban, to be used at dose less than the dose when it is used alone for inhibiting blood coagulation.

As demonstrated in Example 1, co-administration of amiodarone increased the plasma concentration of betrixaban by about 2.5-2.7 fold at 12 hours after administration of betrixaban. Likewise, Example 2 shows that ketoconazole (at 200 mg per day), another Pgp inhibitor, increased betrixaban AUC_{0-∞} by about 2.2 fold and Cₘₐₓ by about 2.4 fold. The effect of ketoconazole is slightly less profound than that of amiodarone even though ketoconazole is a stronger Pgp inhibitor. Despite such slight difference, however, these data demonstrate the synergy between bextrixaban and Pgp inhibitors.

Based on these results, it is contempated that Pgp plays a role in the clearance of betrixaban. Treatment of a subject with a Pgp inhibitor, therefore, reduces the clearance of betrixaban and thus increases its exposure, allowing for more effectiev anticoagulation at a lower dose.

Further, Example 3 provide data to show the synergism between verapamil, another Pgp inhibitor, and betrixaban. Patients receiving both betrixaban and verapamil (240 mg per day) showed 2.9-3.0 folds increase of AUC_{0-∞} for betrixaban than those receiving betrixaban alone. In the same vein, patients receiving both betrixaban and verapamil showed 4.5-4.7 folds increase of Cₘₐₓ compared to those receiving betrixaban alone.

On the one hand, this further confirms the synergism that Pgp inhibitors have on betrixaban. On the other hand, however, such a result was unexpected because ketoconazole and amiodarone are both believed to be stronger Pgp inhibitors than verapamil based on in vitro data. The results indicate that moderate Pgp inhibitors may have larger than anticipated effects on Cₘₐₓ, and that in vitro potency of Pgp inhibition may not enable adequate prediction of potential with betrixaban.

It is also noted that in Example 3, betrixaban and verapamil were co-administered concurrently, while in Example 2 betrixaban was administered 1 hour after ketoconazole. Further, in Example 1, betrixaban was dosed 2 hours after the evening meal while amiodarone was administered at bed time or on the next morning. It is therefore contemplated that the timing of dosing a Pgp inhibitor relative to betrixaban administration also contributes to the magnitude of the effect. In one embodiment, concurrent administration leads to higher synergism than separate administration.

Further, the difference may be attributed to the different permeability and/or solubility of the inhibitors. It is noted that verapamil (BCS class I), ketoconazole (BCS class II), and amiodarone (BCS class II), are all high permeability compounds, while verapamil is also of high solubility. Finally, the specific PK profile of the inhibitor may impact the drug-drug interaction results. In the case of verapamil an extended release formulation (verapamil SR) was used.

By contrast, as reported in Example 4, co-administration with digoxin, a Pgp substrate not inhibiting the activity of Pgp, did not alter the exposure of betrixaban significantly. It is noted that many Pgp inhibitors, including amiodarone and verapamil, are also Pgp substrates, but not all Pgp substrates inhibit the activity of Pgp.

The different effects between digoxin and the Pgp inhibitors therefore further confirm that the synergism between the Pgp inhibitors and betrixaban arises from the inhibition of Pgp, which is involved in the clearance of betrixaban. Nevertheless, the exact effect of each individual Pgp inhibitor, as the unexpected data in the examples demonstrate, may be different.

Thus, one embodiment of the present disclosure provides a method for treating thrombosis or inhibiting blood coagulation in a patient receiving administration of a P-glycoprotein inhibitor, the method comprising administering to the patient a subtherapeutic dose of betrixaban.

In one embodiment, the amount of betrixaban administered is about 20% less than the therapeutically effective amount. In one embodiment, the amount of betrixaban administered is about 50% less than the therapeutically effective amount. Alternatively, the amount of betrixaban administered is about 25%, 30%, 35%, 40%, 45%, 50%, 55%, 60%, 65%, 70%, 75%, 80%, 85% or 90% less than the therapeutically effective amount.

A therapeutically effective amount of betrixaban, depending on the patient and/or condition, such as body weight, of the patient, can be about 40 mg, 60 mg, 80 mg, 90 mg, 110 mg, 130 mg, or 150 mg aggregate daily dose. In a particular aspect, the aggregate daily betrixaban dose for a human patient is about 40 mg. In another aspect, the aggregate daily betrixaban dose for a human patient is about 60 mg. In yet another aspect, the aggregate daily betrixaban dose for a human patient is about 80 mg.

Accordingly, in any one of the above embodiments, the amount of betrixaban administered is from about 25 to about 35 mg. In another embodiment, the amount of betrixaban administered is from about 20 to about 35 mg. In another embodiment, the amount of betrixaban administered is from about 15 to about 35 mg. In another embodiment, the amount of betrixaban administered is from about 10 to about 35 mg. In another embodiment, the amount of betrixaban administered is from about 25 to about 30 mg. In another embodiment, the amount of betrixaban administered is from about 15 to about 30 mg. In another embodiment, the amount of betrixaban administered is from about 10 to about 30 mg. In another embodiment, the amount of betrixaban administered is from about 15 to about 20 mg. In another embodiment, the amount of betrixaban administered is from about 10 to about 20 mg. In another embodiment, the amount of betrixaban administered is from about 10 to about 15 mg.

In some aspects, betrixaban is administered to the patient once daily or twice daily.

In some aspects, the patient receives concomitant administration of the Pgp inhibitor and betrixaban. In a particular aspect, the administration is concurrent.

As used herein and defined above, concomitant administration is intended to mean that during a treatment period, the patient is administered both a factor Xa inhibitor, e.g., betrixaban, and a Pgp inhibitor. They may be administered in the form of two separate pharmaceutical compositions in any form that the agents may be administered alone, for example, one agent is administered orally and the other is administered parenterally. They may be administered at the same time or sequentially in any order. Preferably, when administered sequentially, the two agents are administered sufficiently closely in time such that the desired therapeutic effect can be maximized. In some embodiments, the Pgp inhibitor and betrixaban are administered within about 48 hours, 24 hours, 12 hours, 8 hours, 4 hours, 2 hours, or 1 hour of administration of each other. The two agents may be administered under different dosing schedules. For example, one agent may be administered once a day, and the other may be administered twice a day.

One particular example of concommitant administration is concurrent administration. Therefore, in one aspect, betrixaban and the Pgp inhibitor may be administered in the form of a single pharmaceutical composition which is described in details herein. Alternatively, betrixaban and the Pgp inhibitor may be administered to the same patient within about 10 minutes, 20 minutes, 30 minutes, 40 minutes, 50 minutres, or 60 minutes from each other.

In some embodiments, betrixaban is administered to patients with prior Pgp inhibitor treatment, which is intended to mean that the patient is no longer treated with a Pgp inhibitor after betrixaban treatment commences. Preferably, the prior Pgp inhibitor treatment is sufficiently close in time to the treatment with betrixaban so that the benefit of the prior Pgp inhibitor exposure can be maximized. In some embodiments, the patient's last treatment with a Pgp inhibitor is about or less than one year or six months prior to the commencement of the treatment with a betrixaban. In some embodiments, the patient's last treatment with a Pgp inhibitor is about or less than one month prior to the commencement of the treatment with a betrixaban. In some embodiments, the patient's last treatment with a Pgp inhibitor is about or less than 3 weeks, 2 weeks or 1 week prior to the commencement of the treatment with a betrixaban. In some embodiments, the patient's last treatment with a Pgp inhibitor is about or less than 6 days, 5 days, 4 days, 3 days, 2 days, or 1 day prior to the commencement of the treatment with a betrixaban.

In any of the above embodiments, the patient can receive administration of an therapeutically effective amount or a subtherapeutic dose of the P-glycoprotein inhibitor. In some aspects, the Pgp inhibitor is administered in the form of controlled release.

The examples have demonstrated the safety of co-administration of Pgp inhibitors and betrixaban to human patients. It is further contemplated, however, for patients that are particularly susceptible to side effects of either Pgp inhibitor or betrixaban, it is advisable to avoid the betrixaban treatment during a treatment with a Pgp inhibitor.

Therefore, in one embodiment, the disclosure also provides a method for treating thrombosis or inhibiting blood coagulation, the method comprising administering to the patient a synergistically effective amount of betrixaban, wherein the patient is not currently under treatment with a P-glycoprotein inhibitor. In some aspects, the patient has a history of suffering a side effect of an anti-coagulation therapy. In some aspects, the patient has impaired drug efflux or clearance capabilities.

The methods are useful in treating disease states in mammals which have disorders related to coagulation such as in the treatment or prevention of unstable angina, refractory angina, myocardial infarction, transient ischemic attacks, thrombotic stroke, embolic stroke, disseminated intravascular coagulation including the treatment of septic shock, deep venous thrombosis in the prevention of pulmonary embolism or the treatment of reocclusion or restenosis of reperfused coronary arteries. Further, these compounds are useful for the treatment or prophylaxis of those diseases which involve the production and/or action of factor Xa/prothrombinase complex. This includes a number of thrombotic and prothrombotic states in which the coagulation cascade is activated which include but are not limited to, deep venous thrombosis, pulmonary embolism, myocardial infarction, stroke, thromboembolic complications of surgery and peripheral arterial occlusion. Other diseases treatable or preventable by the administration of compounds of this invention include, without limitation, occlusive coronary thrombus formation resulting from either thrombolytic therapy or percutaneous transluminal coronary angioplasty, thrombus formation in the venous vasculature, disseminated intravascular coagulopathy, a condition wherein there is rapid consumption of coagulation factors and systemic coagulation which results in the formation of life-threatening thrombi occurring throughout the microvasculature leading to widespread organ failure, hemorrhagic stroke, renal dialysis, blood oxygenation, and cardiac catheterization.

With respect to the venous vasculature, abnormal thrombus formation characterizes the condition observed in patients undergoing major surgery in the lower extremities or the abdominal area who often suffer from thrombus formation in the venous vasculature resulting in reduced blood flow to the affected extremity and a predisposition to pulmonary embolism. Abnormal thrombus formation further characterizes disseminated intravascular coagulopathy which commonly occurs within both vascular systems during septic shock, certain viral infections and cancer, a condition wherein there is rapid consumption of coagulation factors and systemic coagulation which results in the formation of life-threatening thrombi occurring throughout the microvasculature leading to widespread organ failure.

In some embodiments, the methods are useful in treating thromboembolic stroke, ischemic or hemorrhagic stroke, systemic embolism, stroke prevention in atrial fibrillation (SPAF), non-valvular atrial fibrillation, venous thromboembolism (VTE), prevention of VTE in knee or hip surgery, prevention of VTE in acute medically ill patients, and secondary prevention in acute coronary syndrome (ACS).

In some embodiments, the methods are for treatment of embolic stroke, thrombotic stroke, venous thrombosis, deep venous thrombosis, acute coronary syndrome, or myocardial infarction.

In some embodiments, the methods are for prevention of stroke in atrial fibrillation patients; prevention of thrombosis in medically ill patients; prevention and treatment of deep vein thrombosis; prevention of arterial thrombosis in acute coronary syndrome patients; and/or secondary prevention of myocardial infarction, stroke or other thrombotic events in patients who have had a prior event.

In some embodiments, the patient has atrial fibrillation. In some embodiments, the patient is a patient with non-valvular atrial fibrillation. In some embodiments, the patient has atrial flutter.

### 3. BETRIXABAN, ITS SALTS AND CRYSTALLINE POLYMORPH FORMS

Betrixaban has the chemical name of [2-({4-[(dimethylamino)iminomethyl]phenyl}carbonylamino)-5-methoxyphenyl]-N-(5-chloro(2-pyridyl))carboxamide and has been disclosed as Example 206 in U.S. Pat. Nos. 6,376,515 and 6,835,739, both of which are incorporated by reference in their entirety herein. Further descriptions of salts and polymorphs of salts of betrixaban can be found in U.S. Pat. No. 7,598,276, which is incorporated by reference in its entirety herein.

In a specific embodiment, the salt of betrixaban is a maleate salt. The maleate salt be formed by protonating one or more nitrogen atoms of betrixaban. In one embodiment, the amidino nitrogen (═NH) of betrixaban is protonated (═NH₂⁺) to form the salt. In some embodiments, the aggregate daily dose of the factor Xa inhibitor is 30 mg of betrixaban and in some embodiments, the 30 mg of betrixaban is administered in the form of a salt, for example the maleate salt.

In one embodiment, the maleate salt of betrixaban is represented by Formula I:

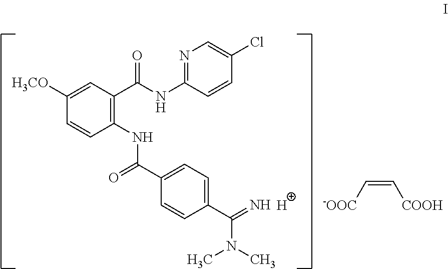

This is also referred to herein as betrixaban maleate. In one embodiment, the aggregate daily dose is about 40 mg of betrixaban maleate. In another embodiment, the aggregate daily dose is about 60 mg, 80 mg, 90 mg, 110 mg, 130 mg, or 150 mg aggregate of betrixaban maleate.

In another embodiment, the salt of betrixaban has a crystalline polymorph form. In some embodiments, the crystalline polymorph of betrixaban maleate is Form I which exhibits a powder X-ray diffraction pattern having at least four and more preferably eight of the following approximate characteristic peak locations: 4.9, 9.7, 13.8, 14.1, 15.2, 17.6, 18.5, 20.8, 21.6, 22.7, 24.1, 26.3, 26.8 degrees 2θ. In still another embodiment, the powder X-ray diffraction pattern has approximate characteristic peak locations of 4.9, 9.7, 11.8, 13.8, 14.1, 15.2, 17.6, 18.5, 19.9, 20.8, 21.6, 22.7, 24.1, 25.0, 26.3, 26.8 degrees 2θ. Form I is further described in U.S. Pat. No. 7,598,276, which is incorporated by reference in its entirety herein. In some embodiments, Form I has a melting point of 201° C.

In some embodiments, the maleate salt of betrixaban is in a crystalline polymorph Form II. In some embodiments, Form II is an anhydrate. In one embodiment, the crystalline polymorph Form II exhibits an X-ray powder diffraction pattern having the following approximate characteristic peak locations: 5.0, 9.7, 10.1, 15.3, 17.5, and 19.6 degrees 2θ. In another embodiment, the X-ray powder diffraction pattern has at least four, six, eight or ten of the approximate characteristic peak locations of 5.0, 9.7, 10.1, 14.6, 15.3, 17.5, 18.0, 18.7, 19.2, 19.6, 22.0, 22.6, 23.0, 23.7, 24.5, 26.5, 26.9, 29.2, 29.5, 30.4 and 35.0 degrees 2θ. In another embodiment, the X-ray powder diffraction pattern has at least four, six, eight or ten of the approximate characteristic peak locations of 5.0, 9.5, 9.7, 10.1, 14.6, 15.3, 17.5, 18.0, 18.7, 19.2, 19.6, 22.0, 22.6, 23.0, 23.7, 24.5, 26.5, 26.9, 29.2, 29.5, 30.4 and 35.0 degrees 2θ. In another embodiment, the X-ray powder diffraction pattern has at least four, six, eight or ten of the approximate characteristic peak locations of 15.3, 5.0, 10.1, 17.5, 9.7, 19.6, 24.5, 18.6, 18.0, 14.5, 22.6, 22.9, 23.0, 22.1, 29.2, 26.5, 24.8, 18.3, and 21.6 degrees 2θ. It is contemplated that the approximate characteristic peaks will have a deviation of up to about 0.1 or 0.05 degrees 2θ.

In another embodiment, the betrixaban maleate salt is in a crystalline polymorph Form III. In some embodiments, Form III exhibits an X-ray powder diffraction pattern having at least the following approximate characteristic peak locations 15.1, 2.2, 4.9, 17.4, 10.0, and 22.4 degrees 2θ. In one embodiment, the X-ray powder diffraction pattern is characterized with peaks having a relative intensity of 10% or more: 15.1, 2.2, 4.9, 17.4, 10.0, 22.4, 26.5, and 2.9 degrees 2θ. In another embodiment, the X-ray powder diffraction pattern has at least six or eight, or ten, or all of the approximate characteristic peak locations selected from 15.1, 2.2, 4.9, 17.4, 10.0, 22.4, 26.5, 2.9, 24.6, 19.4, 24.2, 16.3, 20.7, 22.9, 29.0, 9.6, 18.0, 18.5, 29.3, 22.0, and 30.3 degrees 2θ. In another embodiment, the X-ray powder diffraction pattern has at least four, six, eight, ten or all of the approximate characteristic peak locations of 15.1, 2.2, 4.9, 17.4, 10.0, 22.4, 26.5, 2.9, 24.6, 19.4, 24.2, 16.3, 20.7, 22.9, 29.0, 9.6, 18.0, 18.5, and 29.3 degrees 2θ.

In some embodiments, Form III is a hydrate. In some embodiments, Form III is a hemihydrate. In some embodiments, the Form III is channel hydrate.

Betrixaban can be prepared according to methods described in U.S. Pat. Nos. 6,376,515 and 7,598,276, and U.S. patent application Ser. No. 12/969,371, filed Dec. 15, 2010, all of which are hereby incorporated by reference in their entirety. Preparation of the maleate salt of betrixaban and Form I is described in U.S. Pat. No. 7,598,276.

Form II can be prepared by dissolving betrixaban maleate salt (which may be in the polymorph Form I) in a solvent at a temperature which is above room temperature but below the boiling point of the solvent (for example about 50-70° C.), optionally followed by addition of a seed of Form II to ensure that Form II grows, and cooling the solution slowly (for example to 0° C. over 16 hours). In some embodiments, the solvent comprises an anhydrous solvent such as, e.g., dry ethanol. In some embodiments, the solvent comprises water. The ratio of the ethanol to water in the solvent may vary. In specific embodiments, the ratio can be up to about 1:1, for example from about 1:3 to 1:1. Other solvents can be used include tetrahydrofuran, methyl tert-butyl ether, dimethylformamide, and toluene, for example, mixtures of tetrahydrofuran/water, methyl tert-butyl ether/dimethylformamide, and toluene/dimethylformamide. Form I is favored when supersaturation is high and nucleation dominates under less-controlled process. Form II is favored when there is adequate Form II seed and the crystallization is slow enough that growth dominates over nucleation.

In some embodiments, crystalline polymorph Form II can be prepared by a method comprising heating betrixaban maleate salt in a solvent comprising water and optionally ethanol to a temperature of at least about 50° C. to obtain a solution, and cooling the solution to at or below about 20° C. but above the freezing temperature of the solvent.

In some embodiments, the method comprises heating a composition comprising betrixaban free base and at least one equivalent of maleic acid in a solvent comprising water and optionally ethanol to a temperature of about 45° C. to about 60° C., addition of a seed crystal of From II, and cooling the solution to at or below about 30° C. but above the freezing temperature of the solvent. In some embodiments, the solvent comprises water and ethanol in a volume ratio of about 65:35.

The polymorph Form III can be prepared by recrystallizing the maleate salt in a suitable solvent in which betrixaban maleate is completely or partially soluble at a desired temperature. In some embodiments, the solvent comprises greater than 25% of water, such as a solvent comprising 25% ethanol and 75% water. Other solvents can be used include tetrahydrofuran, methyl tert-butyl ether, dimethylformamide, and toluene, for example, mixtures of tetrahydrofuran/water, methyl tert-butyl ether/dimethylformamide, and toluene/dimethylformamide. In some embodiments, Form III is formed in such a solvent at a temperature that is higher than room temperature, for example, at about 60° C. The hemihydrate Form III may be converted to the anhydrous polymorph Form II when it is dried and/or crushed. The anhydrous polymorph Form II may be converted to the hemihydrate Form III when it is exposed to a relative humidity of greater than 25%.

More detailed descriptions and methods of preparing Form II and Form III can be found in U.S. patent application Ser. No. ______, Attorney Docket No: 099202-3001, entitled “Crystalline polymorphs of a factor Xa inhibitor” concurrently filed with this application, the contents of which are hereby incorporated by reference in their entirety into the present disclosure.

### 4. P-GLYCOPROTEIN INHIBITORS

P-glycoprotein inhibitors are generally known, including but not limited to, amiodarone, ketoconazole, clarithromycin, verapamil, diltiazem, cyclosporine, quinidine, erythromycin, itraconazole, ivermectin, mefloquine, nifedipine, ofloxacin, propafenone, ritonavir, tacrolimusvalspodar (PSC-833), zosuquidar (LY-335979), elacridar (GF120918), HM30181AK, R101933, and R102207, or a pharmaceutically acceptable salt thereof.

In some embodiments, the P-glycoprotein inhibitor is selected from the group consisting of amiodarone, ketoconazole and verapamil.

The effective amount of the Pgp inhibitor is an amount effective to inhibit coagulation and/or treat thrombosis when administered in combination with the factor Xa inhibitor. It is contemplated that in some embodiments, the effective amount of the Pgp inhibitor in the combination therapy is at an amount of the Pgp inhibitor when used alone. In some embodiments, the effective amount is an amount that is lower than the amount needed to produce the same level of effect when it is used alone, which is referred to as “subtherapeutic dosage.” The effective amount will vary depending upon the specific combination, the subject and disease condition being treated, the weight and age of the subject, the severity of the disease condition, the dosing regimen to be followed, timing of administration, the manner of administration and the like, all of which can be determined readily by one of ordinary skill in the art.

Pgp inhibitors may be associated with a greater risk of hip fractures and Clostridium difficile-associated diarrhea and with an increase in occurrence of pneumonia. In particular, it is recommended that the first few doses of amiodarone, which is for treating and preventing certain types of serious, life-threatening ventricular arrhythmias be administered in a hospital setting as it has the potential to cause side effects that could be fatal. The side effects include certain serious heart conditions, for example, atrioventricular block, faintness, liver disease, asthma or another lung disorder, vision problems, high or low blood pressure, a thyroid disorder, etc. Therefore, a reduced dosage of the P-glycoprotein inhibitor when combined with a factor Xa inhibitor is contemplated to be beneficial in reducing or avoiding these side effects.

In some embodiments, the P-glycoprotein inhibitor is amiodarone. In some embodiments, the amiodarone is administered in a hydrochloride salt form. In some embodiment, amiodarone is administered orally. In some embodiments, amiodarone is administered either once or twice daily. In some embodiments, amiodarone is administered in an amount of about 100 mg to about 600 mg, about 100 mg to about 500 mg, about 100 mg to about 400 mg, about 100 mg to about 300 mg, or about 200 mg to about 400 mg amiodarone or a pharmaceutically acceptable salt thereof. In some embodiments, amiodarone is administered in a tablet form having about 100 mg to about 400 mg, about 100 mg to about 300 mg, or about 200 mg to about 400 mg amiodarone or a pharmaceutically acceptable salt thereof per tablet. In some embodiments, the effective amount of amiodarone is an aggregate daily dose of about 100 or 200 mg, administered either once or twice daily. In some embodiments, the effective amount of amiodarone is an aggregate daily dose of less than 100 or 200 mg, administered either once or twice daily. In some embodiments, a total of 10 grams of amiodarone is administered in divided doses over one to two weeks.

In some embodiments, a loading dose of 800 to 1,600 mg/day amiodarone is administered for a period of about 1 to 3 weeks, or longer until an initial therapeutic response occurs. Loading dose of amiodarone may be over 1000 mg/day by bid or tid dosing. In some embodiments, for example, when adequate arrhythmia control is achieved, or if side effects become prominent, amiodarone is reduced to about 600 to about 800 mg/day for one month and then to a maintenance dose, for example, about 400 to about 600 mg/day. In some embodiments, the maintenance dose of amiodarone is 100 or 200 mg administered once or twice a day.

In some embodiment, amiodarone is administered intravenously. In some embodiments, the effective amount of amiodarone is a loading dose of about 300 mg in a 20-30 mL solution or 150 mg in a 100 mL solution administered over 10 minutes. In some embodiments, the loading dose is followed by a 360 mg slow infusion over 6 hours and then a maintenance infusion of 540 mg over 18 hours.

In some embodiments, amiodarone or a pharmaceutically acceptable salt, for example, hydrochloric acid salt, is administered at the following dosing regime:

Loading infusions. about 1000 mg over the first 24 hours of therapy, delivered by the following infusion regimen:
first Rapid infusion of 150 mg over the first 10 minutes at 15 mg/min;
followed by slow infusion of 360 mg over the next 6 hours at 1 mg/min); and maintenance infusion of 540 mg over the remaining 18 hours at 0.5 mg/min.

After the first 24 hours, the maintenance infusion rate of 0.5 mg/min (720 mg/24 hours) using a concentration of 1 to 6 mg/mL, which may be continued for 2 to 3 weeks

In some embodiments, the P-glycoprotein inhibitor is ketoconazole. In some embodiment, ketoconazole is administered orally. In some embodiments, ketoconazole is administered either once or twice daily. In some embodiments, ketoconazole is administered in an amount of about 100 mg to about 600 mg, about 100 mg to about 500 mg, about 100 mg to about 400 mg, about 100 mg to about 300 mg, or about 200 mg to about 400 mg ketoconazole or a pharmaceutically acceptable salt thereof. In some embodiments, ketoconazole is administered in a tablet form having about 100 mg to about 400 mg, about 100 mg to about 300 mg, or about 200 mg to about 400 mg ketoconazole or a pharmaceutically acceptable salt thereof per tablet. In some embodiments, the effective amount of ketoconazole is an aggregate daily dose of about 200 or 400 mg, administered either once or twice daily. In some embodiments, the effective amount of ketoconazole is an aggregate daily dose of less than 200 or 400 mg, administered either once or twice daily.

In some embodiments, ketoconazole is administered topically as a cream. The ketoconazole cream, in some embodiments, is about 1%, 2%, or 4% to be applied once daily to cover the affected and immediate surrounding area on the skin.

In some embodiments, the P-glycoprotein inhibitor is verapamil. In some embodiments, the verapamil is administered in a hydrochloride salt form. In some embodiment, verapamil is administered orally. In some embodiments, verapamil is administered either once or twice daily. In some embodiments, verapamil is administered in an amount of about 20 mg to about 400 mg, about 40 mg to about 300 mg, or about 40 mg to about 200 mg verapamil or a pharmaceutically acceptable salt thereof. In some embodiments, verapamil is administered in a tablet form having about 40 mg to about 200 mg, about 40 mg to about 120 mg, or about 40 mg to about 80 mg verapamil or a pharmaceutically acceptable salt thereof per tablet. In some embodiments, the effective amount of verapamil is an aggregate daily dose of about 100 or 200 mg, administered either once or twice daily. In some embodiments, the effective amount of verapamil is an aggregate daily dose of less than 240 or 360 mg, administered either once or twice or tree times daily. In some embodiments, a total of 10 grams of verapamil is administered in divided doses over one to two weeks.

### 5. FORMULATIONS

Another aspect of the invention provides an aggregate daily dose comprising a factor Xa inhibitor and a P-glycoprotein inhibitor wherein at least one of the factor Xa inhibitor and the P-glycoprotein inhibitor is in a subtherapeutic dose. Another aspect of the invention provides an aggregate daily dose comprising a factor Xa inhibitor in an amount of about 10 to about 20 mg and an effective amount a P-glycoprotein inhibitor. The factor Xa inhibitor, the Pgp inhibitor and the effective amount of the Pgp inhibitor are as described herein. In some embodiments, the amount of the factor Xa inhibitor is an aggregate daily dose of about 10, 15, 20, 25, 30, 35, or 40 mg. In some embodiments, the aggregate daily dose is formulated for administration to the patient once or twice daily.

In some embodiments, the unit dose formulation further comprises a pharmaceutically acceptable carrier.

The compositions of this invention may be in the form of tablets, capsules, lozenges, or elixirs for oral administration, suppositories, sterile solutions or suspensions or injectable administration, and the like, or incorporated into shaped articles. The method of administration will vary from subject to subject and be dependent upon such factors as the type of mammal being treated, its sex, weight, diet, concurrent medication, overall clinical condition, the particular compounds and/or salts employed, the specific use for which these compounds and/or salts are employed, and other factors which those skilled in the medical arts will recognize.

Capsules useful in the present invention can be prepared using conventional and known encapsulation techniques, such as that described in Stroud et al., U.S. Pat. No. 5,735,105. The capsule is typically a hollow shell of generally cylindrical shape having a diameter and length sufficient so that the pharmaceutical solution compositions containing the appropriate dose of the active agents fit inside the capsule. The exterior of the capsules can include plasticizer, water, gelatin, modified starches, gums, carrageenans, and mixtures thereof. Those skilled in the art will appreciate what compositions are suitable.

In addition to the active agents, tablets useful in the present invention can comprise fillers, binders, compression agents, lubricants, disintegrants, colorants, water, talc and other elements recognized by one of skill in the art. The tablets can be homogeneous with a single layer at the core, or have multiple layers in order to realize preferred release profiles. In some instances, the tablets of the instant invention may be coated, such as with an enteric coating. One of skill in the art will appreciate that other excipients are useful in the tablets of the present invention.

Lozenges useful in the present invention include an appropriate amount of the active agents as well as any fillers, binders, disintegrants, solvents, solubilizing agents, sweeteners, coloring agents and any other ingredients that one of skill in the art would appreciate is necessary. Lozenges of the present invention are designed to dissolve and release the active agents on contact with the mouth of the patient. One of skill in the art will appreciate that other delivery methods are useful in the present invention.

Formulations of this invention are prepared for storage or administration by mixing active agents having a desired degree of purity with physiologically acceptable carriers, excipients, stabilizers etc., and may be provided in sustained release or timed release formulations. Acceptable carriers or diluents for therapeutic use are well known in the pharmaceutical field, and are described, for example, in Remington's Pharmaceutical Sciences, Mack Publishing Co., (A.R. Gennaro Ed. 1985). Such materials are nontoxic to the recipients at the dosages and concentrations employed, and include buffers such as phosphate, citrate, acetate and other organic acid compounds and/or salts, antioxidants such as ascorbic acid, low molecular weight (less than about ten residues) peptides such as polyarginine, proteins, such as serum albumin, gelatin, or immunoglobulins, hydrophilic polymers such as polyvinylpyrrolidinone, amino acids such as glycine, glutamic acid, aspartic acid, or arginine, monosaccharides, disaccharides, and other carbohydrates including cellulose or its derivatives, glucose, mannose or dextrins, chelating agents such as EDTA, sugar alcohols such as mannitol or sorbitol, counterions such as sodium, and/or nonionic surfactants such as Tween, Pluronics or polyethyleneglycol.

Preferably, dosage formulations of the invention to be used for therapeutic administration are sterile. Sterility is readily accomplished by filtration through sterile membranes such as 0.2 micron membranes, or by other conventional methods. Formulations typically will be stored in lyophilized form or as an aqueous solution. The pH of the preparations of this invention typically will be between 3 and 11, more preferably from 5 to 9 and most preferably from 7 to 8. It will be understood that use of certain of the foregoing excipients, carriers, or stabilizers may result in the formation of cyclic polypeptide compounds and/or salts. Route of administration may be by injection, such as intravenously (bolus and/or infusion), subcutaneously, intramuscularly, or colonically, rectally, nasally or intraperitoneally. Other dosage forms such as suppositories, implanted pellets or small cylinders, aerosols, oral dosage formulations (such as tablets, capsules and lozenges) and topical formulations such as ointments, drops and dermal patches may be used. The sterile membranes may be desirably incorporated into shaped articles such as implants which may employ inert materials such as biodegradable polymers or synthetic silicones, for example, Silastic, silicone rubber or other polymers commercially available.

The compositions of this invention may be in the form of liposome delivery systems, such as small unilamellar vesicles, large unilamellar vesicles and multilamellar vesicles. Liposomes can be formed from a variety of lipids, such as cholesterol, stearylamine or phosphatidylcholines.

The compositions of this invention may also be delivered by the use of antibodies, antibody fragments, growth factors, hormones, or other targeting moieties, to which the salt molecules are coupled. The compositions of this invention may also be coupled with suitable polymers as targetable drug carriers. Such polymers can include polyvinylpyrrolidinone, pyran copolymer, polyhydroxy-propyl-methacrylamide-phenol, polyhydroxyethyl-aspartamide-phenol, or polyethyleneoxide-polylysine substituted with palmitoyl residues. Furthermore, compositions of the invention may be coupled to a class of biodegradable polymers useful in achieving controlled release of a drug, for example polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Polymers and semipermeable polymer matrices may be formed into shaped articles, such as valves, stents, tubing, prostheses and the like.

In some embodiments, an amiodarone tablet comprises amiodarone hydrochloride, lactose monohydrate, magnesime stearate, povidone, pregelatinized corn starch, sodium starch glycolate, steric acid, and opotionally one or more coloring agents.

### 6. EXAMPLES

The materials in the examples are generally known, which may be prepared by conventional means or available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5^{th} Edition, 2001), and Larock's Comprehensive Organic Transformations (VCH Publishers Inc., 1989).

Unless stated otherwise, the abbreviations used throughout the specification have the following meanings.
AUC=area under curve
CI=confidence interval
DDI=drug-drug interaction
G=gram
GMR=geometric least-squares mean ratio
Hr=hour
LS=least square
L=liter
M=molar
Mg=milligram
mL=milliliter
nM=nanomolar
μM=micromolar
PK=pharmacokinetics
SD=standard deviation

### Example 1

### Amiodarone Increases the Plasma Concentrations of Betrixaban

This example demonstrates that plasma concentrations of betrixaban are significantly increased by co-administration with amiodarone.

A clinical trial was conducted to determine the antithrombosis potential of betrixaban in a target population for stroke prevention in atrial fibrillation (SPAF). Patients were divided into three groups and were administered with a once daily oral dose of betrixaban of 40, 60, or 80 mg, respectively, for a minimum of 12 weeks. In each dosage group, some patients were also administered with amiodarone.

Betrixaban was dosed two hours after evening meal and amiodarone was typically dosed in the morning. Dosage of amiodarone for each individual patient was individualized based on each patient's health condition and need, but was in the range of 200 mg per day to 600 mg per day as maintenance doses and 800 mg per day to 1600 mg per day as loading doses for 1 to 3 weeks. Electrocardiogram (ECG) can be used for dose titration.

Inclusion criteria for patient intake included:
requires long term anticoagulation for stroke prevention in atrial fibrillation;
current non-valvular atrial fibrillation or atrial flutter or electrocardiogram (EGG) or Holter documentation within the past 12 months.

Exclusion criteria for patient intake include:
age under 18;
pregnant or planning to become pregnant;
routinely consumes more than 2 alcoholic drinks per day (average >14 alchoholic drinks per week) or greater than 5 drinks within 2 hours on accasion;
major surgery in the past month;
surgery or intervention planned in the next 3 months;
intracranial, intraocular, spinal, retroperitoneal or atraumatic intra-auricular bleeding within 6 months;
gastrointerstinal bleeding within 90 days;
symptomatic or endoscopically documented gastroduodenal ulcer disease within 30 days;
hemorrhagic disorder or bleeding diathesis;
liver disease;
uncontrolled hypertension;
active bleeding;
conditions that requires chronic anticoagulation (other than atrial fibrillation);
severe aortic and mitral valvular disease requiring surgical intervention;
history of coagulopathy;
active infective endocarditis; and
history of familial long QT syndrome.

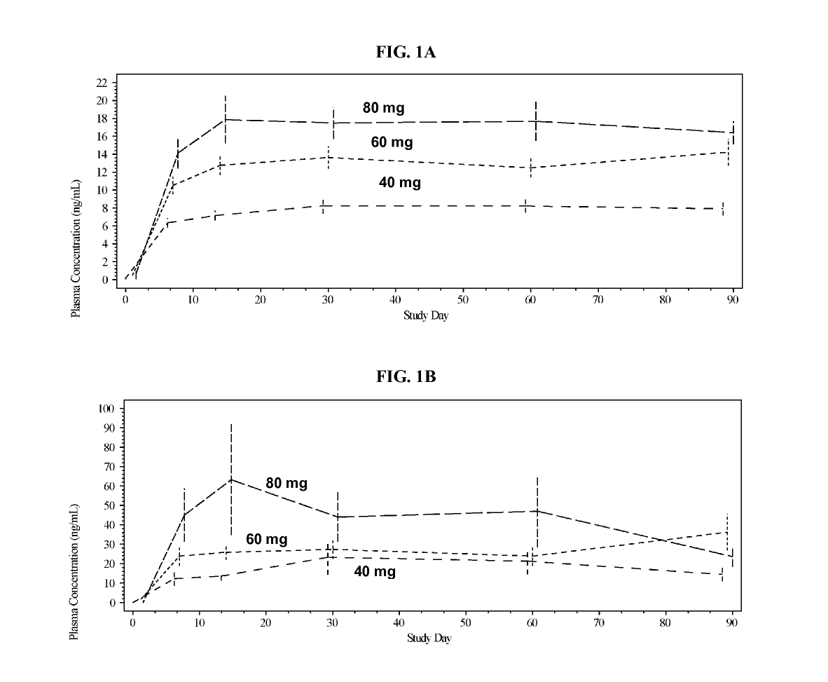

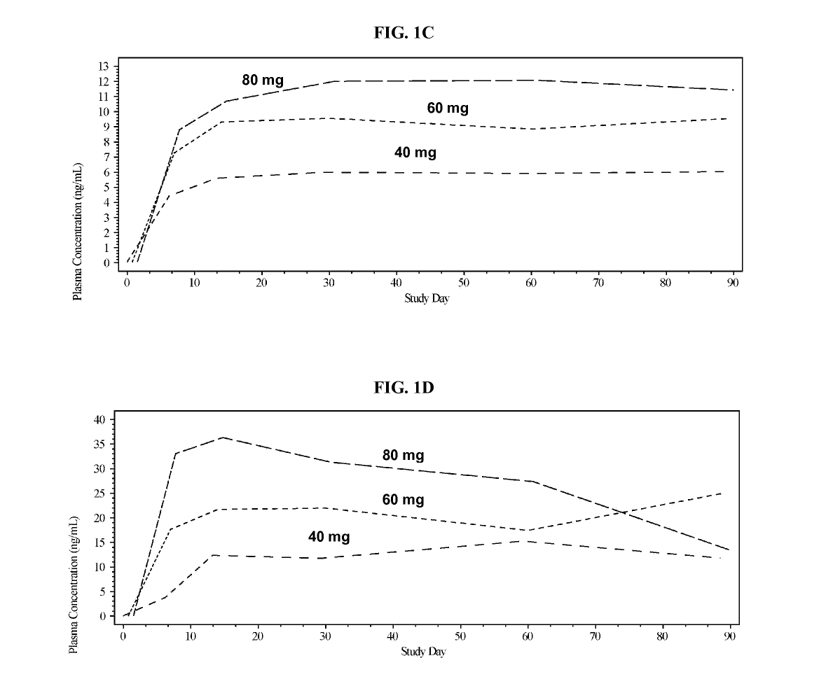

Betrixaban plasma concentrations were determined and shown in FIG. 1A-1D. FIGS. 1A and 1C show the plasma concentrations of betrixaban in patients with betrixaban treatment only and FIGS. 1B and 1D show the plasma concentrations of betrixaban in patients with concomitant betrixaban and amiodarone treatment. These figures demonstrate that plasma concentrations of betrixaban were significantly higher in patients.

For instance, comparing FIGS. 1A and 1B, the maximum betrixaban plasma concentrations for each dosing group were approximately 18 ng/mL vs. 60 ng/mL (80 mg betrixaban without or with concomitant amiodarone treatment), 14 ng/mL vs. 25 ng/mL (60 mg betrixaban without or with concomitant amiodarone treatment), and 8 ng/mL vs. 20 ng/mL (40 mg betrixaban without or with concomitant amiodarone treatment). Such differences were likewise apparent between FIGS. 1C and 1D: 12 ng/mL vs. 36 ng/mL, 9 ng/mL vs. 22 ng/mL and 6 ng/mL vs. 12 ng/mL. The use of amiodarone, therefore, increased the maximum plasma concentrations of betrixaban by about 2-3 folds.

It was also observed that the amiodarone use resulted in an approximately 2.5-2.7 folds increase in betrixaban C₁₂ₕᵣ based on a population pharmacokinetics (POP PK) analysis.

A total of 35 atrial fibrillation patients were on concomitant amiodarone and betrixaban, 9 receiving 40 mg betrixaban, 15 receiving 60 mg betrixaban, and 11 receiving 80 mg betrixaban. The Table shows representative patients with their plasma amiodarone concentrations, which cover a broad range attesting to the feasibility of combination use of betrixaban and amiodarone.

| Pat | | Plasma Concentration | Betrixaban |
|---|---|---|---|
| #ID | Visit | of amiodarone (mg/L) | dose per day |
| A | week 4 | 0.891 | 40 mg |
| B | week 4 | 0.968 | 60 mg |
| C | week 4 | 0.62 | 40 mg |
| D | week 4 | 0.691 | 40 mg |
| E | week 4 | 0.432 | 60 mg |
| F | week 4 | 0.607 | 60 mg |
| H | week 4 | 0.671 | 40 mg |
| I | week 4 | 0.555 | 60 mg |
| J | week 4 | 1.349 | 60 mg |
| K | week 4 | 0.542 | 80 mg |

Therefore, this example shows that amiodarone increases the exposure of betrixaban indicating that a lower dosage of betrixaban can be used to achieve similar therapeutic effect when a patient is concomitantly treated with amiodarone. Conversely, for a patient that is susceptible to potential adverse effects of betrixaban, a lowered dose or even avoidance of betrixaban is warranted.

### Example 2

### Ketoconazole Increases the Plasma Concentrations of Betrixaban

The example demonstrates that co-administration of ketoconazole affects the pharmacokinetic profile of betrixaban.

### Methods

This example uses a single-center, open-label, randomized sequence, 2-way crossover study of a single dose of betrixaban administered to 12 healthy subjects on 2 occasions, once alone and once following 5 days of ketoconazole 200 mg administered orally every 12 hours. There was a 12- to 14-day washout period between the two administrations of betrixaban. Blood and urine samples were obtained at specific time intervals after dosing for pharmacokinetic evaluations.

Subjects received betrixaban maleate capsules 40 mg (as free base) (from Portola Pharmaceuticals, Inc.). Ketoconazole (200 mg tablets) was obtained from Astra Zeneca.

For each subject, the total duration of the study was up to 11 weeks (4 weeks predose, 1 week in the study unit on 2 occasions separated by a 12- to 14-day washout period, and 3 weeks after the last dose until the termination visit).

### Pharmacokinetics

Pharmacokinetic blood samples for determination of betrixaban were collected predose and 0.5, 1, 2, 3, 4, 6, 8, 12, 18, 24, 36, 48, 60, 72, and 96 hours postdose. Pharmacokinetic blood samples for ketoconazole were collected within 30 minutes predose and at 0.75, 2, 3, 4, 6, 8, and 12 hours postdose on Day-1. On Day 1 blood samples were collected immediately prior to dosing with ketoconazole and at 0.75, 2, 3, 4, 6, 8, and 12 hours post ketoconazole dosing.

Urine was collected for concentrations of betrixaban within 30 minutes prior to dosing on Day 1. Batched urine was collected 0 to 24, 24 to 48, and 48 to 72 hours after dosing. Pharmacokinetic parameters calculated for betrixaban included: area under the concentration-time curve from time zero to infinity [AUC_{(0-∞)}], area under the concentration-time curve from time zero to 72 hours [AUC₍₀₋₇₂₎], area under the concentration-time curve from time zero to time of last measurable concentration [AUC_{(0-Tlast)}], maximum observed plasma concentration (Cₘₐₓ), time to maximum observed plasma concentration (Tₘₐₓ), terminal rate constant (λz), terminal plasma half-life (t_{1/2}), apparent oral clearance (CL/F), apparent volume of distribution (Vz/F), relative bioavailability (Frel), and Ratio of Cₘₐₓ (R).

Pharmacokinetic parameters calculated for ketoconazole included: maximum observed plasma concentration (Cₘₐₓ), time to maximum observed plasma concentration (Tₘₐₓ), area under the concentration-time curve from time zero to 12 hours [AUC₍₀₋₁₂₎], and apparent oral clearance (CL/F).

Pharmacokinetic parameters calculated for betrixaban from urine included: cumulative amount of unchanged drug excreted in urine from time zero to 72 hours [Ae₍₀₋ₜ₎], cumulative fraction of dose excreted unchanged in the urine [fe₍₀₋ₜ₎], and renal clearance (CLr).

### Analytical Methodology

Plasma and urine concentrations of betrixaban and plasma concentrations of ketoconazole were determined by means of validated, sensitive, and specific high performance liquid chromatography/tandem mass spectrometric assays. The lower limits of quantitation of the betrixaban assay method were 0.100 ng/mL for plasma and 0.500 ng/mL for urine. The lower limits of quantitation of the ketoconazole assay method were 20 ng/mL for plasma.

### Safety

Safety was monitored by adverse event monitoring, clinical laboratory testing (hematology, serum chemistry, and urinalysis), vital sign measurements (oral temperature, respiratory rate, pulse rate, and systolic and diastolic blood pressure), electrocardiograms, and physical examinations.

### Statistical Methods

Pharmacokinetic parameters were derived using noncompartmental methods. betrixaban and ketoconazole concentrations were summarized using descriptive statistics for each treatment period. Concentrations below quantitation limit (0.1 ng/ml for betrixaban and 20 ng/mL for ketoconazole) were treated as zero for descriptive statistics.

### Results

Pharmacokinetic blood samples for betrixaban were collected for each treatment group at predose and 0.5, 1, 2, 3, 4, 6, 8, 12, 18, 24, 36, 48, 60, 72, and 96 hours postdose. FIG. 2 presents the mean betrixaban plasma concentration-time profiles by treatment group for all subjects. Mean plasma concentrations of betrixaban were quantifiable up to 96 hours after a single oral dose of betrixaban at 40 mg.

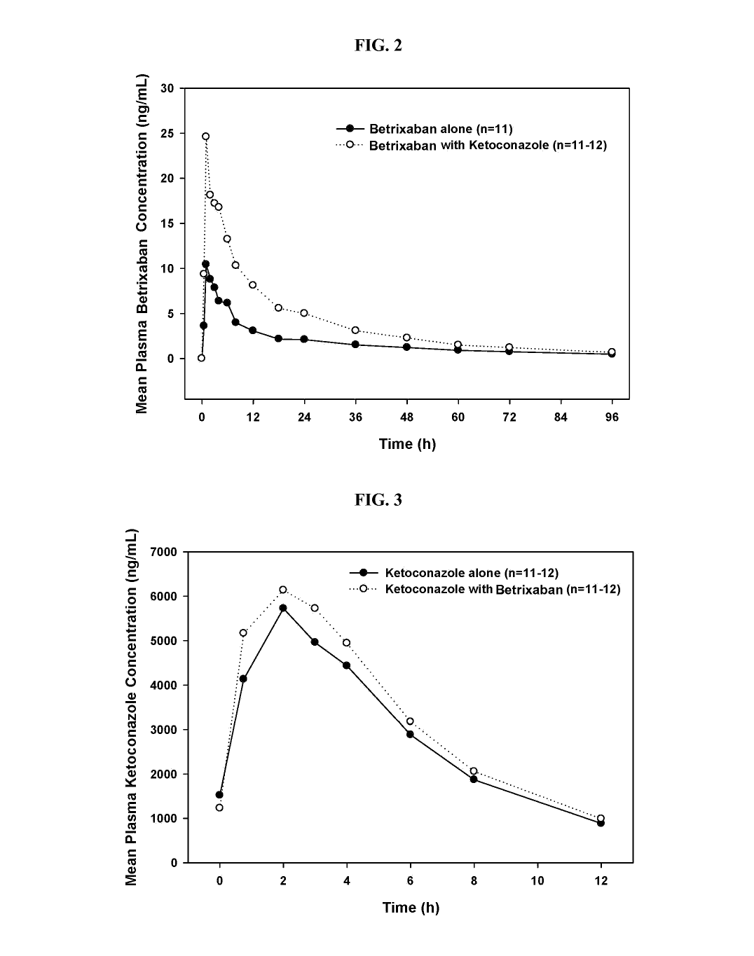

Pharmacokinetic blood samples for ketoconazole were collected on Day-1 and Day 1 (Day 1 was the day of betrixaban dosing) at predose and 0.75, 2, 3, 4, 6, 8, and 12, hours postdose. FIG. 3 presents the mean ketoconazole plasma concentration-time profiles by treatment for all subjects. Mean plasma concentrations of ketoconazole were quantifiable up to 12 hours after a dosing with 200 mg ketoconazole.

There were no deaths or serious adverse effect during study conduct. One subject discontinued but was considered unlikely related to study medication.

### Plasma Pharmacokinetics of Betrixaban

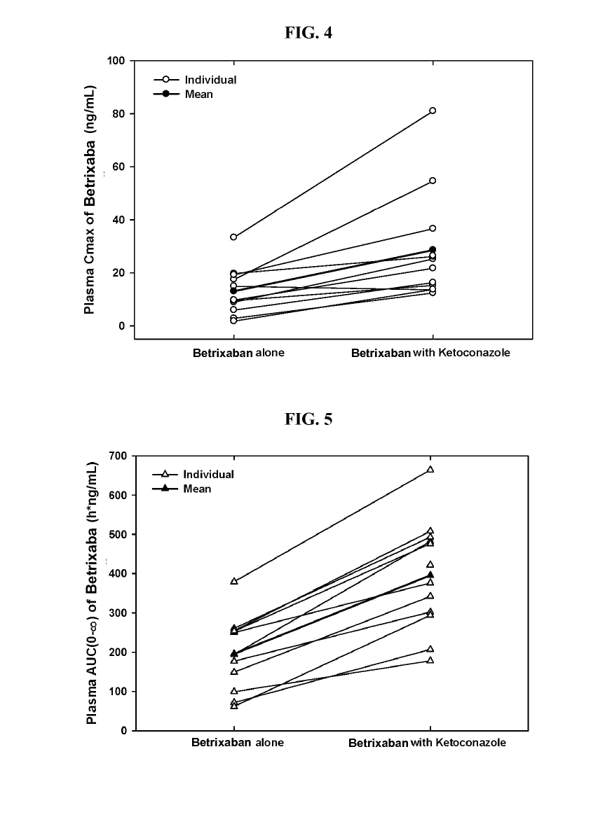

Following a single oral administration of 40 mg of betrixaban alone or betrixaban after 5 days of ketoconazole administration (200 mg every 12 hours), the maximal plasma concentration of betrixaban was reached at 1 hour in both the treatment groups. There was a significant difference in the Cₘₐₓ of betrixaban in the 2 treatment groups with the mean standard deviation (SD) Cₘₐₓ values being 13.01 (9.16) and 28.57 (20.44) ng/mL, respectively, for the betrixaban alone and betrixaban+ketoconazole groups (FIG. 4).

Following Cₘₐₓ, the betrixaban concentration in the plasma declined in a biphasic manner. There was also a significant difference in the terminal elimination half-life of betrixaban in the 2 treatment groups, with the median terminal elimination half-life being 34.5 h and 25.8 h respectively in the betrixaban alone and betrixaban+ketoconazole groups.

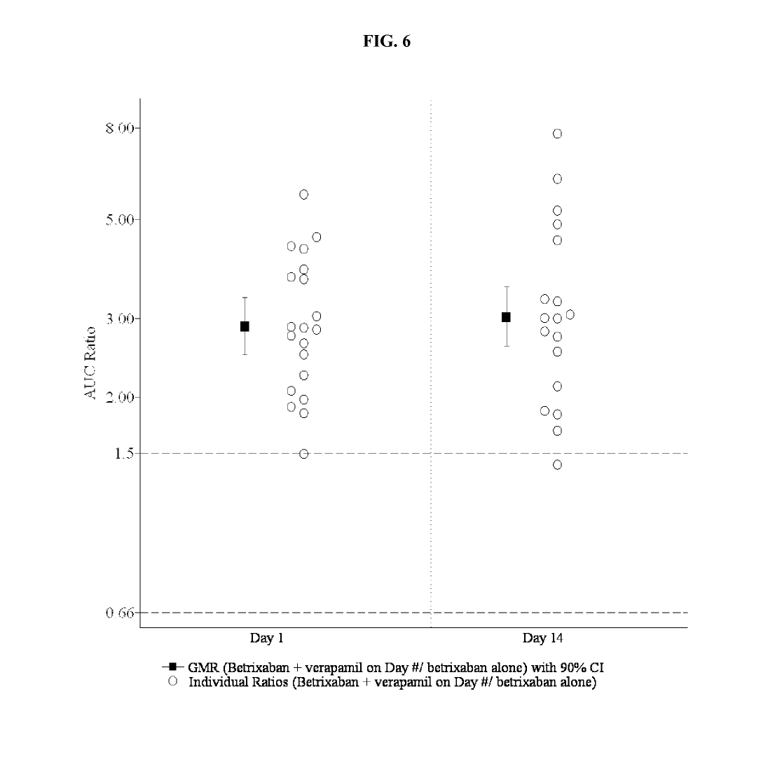

Similar to Cₘₐₓ, differences were seen in the AUC exposure parameters between the 2 treatment groups. For betrixaban alone, the mean (standard deviation (SD)) plasma AUC_{(0-∞)}, AUC_{(0-Tlast)} and AUC₍₀₋₇₂₎ values for betrixaban were 195.4 (96.2), 169.8 (87.5), and 155.1 (81.4) ng*h/mL, respectively. For betrixaban+ketoconazole, the AUC_{(0-∞)}, AUC_{(0-Tlast)}, and AUC₍₀₋₇₂₎ values with the mean (SD) values being 395.3 (139.5), 368.6 (132.6), and 346.4 (125.7) ng*h/mL, respectively. The AUC_{(0-∞)} for betrixaban increased for all subjects (who completed both the treatments) with ketoconazole administration (FIG. 5).

The relative bioavailability calculated as the ratio of least squares geometric mean AUC_{(0-∞)} of betrixaban with ketoconazole/AUC_{(0-∞)} of betrixaban alone was 212%, whereas the ratio of Cₘₐₓ of betrixaban with ketoconazole/betrixaban alone was 234%. Ketoconazole significantly increased the exposure of betrixaban in the subjects, with the 90% confidence interval (CI) of geometric LS mean ratios of Cₘₐₓ, AUC_{(0-∞)}, and AUC_{(0-Tlast)} being totally outside the 80 to 125% limits.

There was also a difference in the CL/F and Vz/F parameters for the 2 treatment groups with the values for CL/F and Vz/F being 60-70% lower for the betrixaban plus ketoconazole group as compared to the betrixaban alone group (FIG. 3).

Table 1 shows the effects of ketoconazole on betrixaban plasma pharmacokinetics and Table 2 is a summary of arithmetic mean (with standard deviation) of key pharmacokinetic parameters by treatment group.

TABLE 1
Effect of ketoconazole on betrixaban plasma pharmacokinetics

| Parameter | | | Geometric | Pairwise Comparisons | | | |
|---|---|---|---|---|---|---|---|
| (unit) | Treatment | n | LS Mean | Pair | Ratio (%) | 90% CI | p-value |
| Cₘₐₓ | A | 11 | 9.996 | B/A | 233.8 | (183.1, 298.4) | 0.0001 |
| (ng/mL) | B | 12 | 23.37 | | | | |
| AUC_{(0-∞)} | A | 11 | 171.1 | B/A | 211.9 | (179.8, 249.6) | <0.0001 |
| (ng*h/mL) | B | 12 | 362.5 | | | | |
| AUC_{(0-Tlast)} | A | 11 | 147.3 | B/A | 228.5 | (191.3, 272.9) | <0.0001 |
| (ng*h/mL) | B | 12 | 366.5 | | | | |
| Tₘₐₓ | A | 11 | 1.00 [a] | | | (0.50, 6.00) [b] | |
| (hr) | B | 12 | 1.00 [a] | | | (0.50, 6.02) [b] | |

Treatment A: 40 mg of betrixaban alone; Treatment B: 40 mg of betrixaban following 5 days of ketoconazole 200 mg orally every 12 hours.
Note:
Based on fitting a linear mixed model with fixed effects for sequence, period, and treatment and a random effect for subject within a sequence to the log-transformed values.
n = number of subjects;
LS = Least-squares;
CI = confidence interval
[a] Tₘₐₓ presented as median
[b] Tₘₐₓ interval presented as range

TABLE 2
Effect of co-administration of betrixaban on key ketoconazole plasma
pharmacokinetics parameters

| | | | PRT054021 | |
|---|---|---|---|---|
| Parameter | PRT054021 Alone | | with Ketoconazole | |
| (unit) | (n = 11) | | (n = 12) | |
| Cₘₐₓ (ng/mL) | 13.01 | (9.16) | 28.57 | (20.44) |
| AUC(0-∞) | 195.4 | (96.2) | 395.3 | (139.5) |
| (ng * h/mL) | | | | |
| Tₘₐₓ (h) [a] | 1 | (0.5-6.0) | 1 | (0.5-6.02) |
| t½ (h) [a] | 34.5 | (29.02-48.73) | 25.76 | (21.13-32.89) |
| CL/F (L/h) | 276.7 | (180.3) | 115.9 | (49.4) |
| Vz/F (L) | 14730 | (10143) | 4398 | (1994) |

Notes:
SD = standard deviation.
[a] Median and range reported

### Plasma Pharmacokinetics of Ketoconazole

Ketoconazole pharmacokinetics were determined on Day-1 and Day 1 of the study with the Day 1 being the day of betrixaban administration. Following ketoconazole administration on Day-1, the median Tₘₐₓ was reached at 2 hours. The mean (SD) Cₘₐₓ achieved was 5902.9 (2463.8) ng/mL. Following Cₘₐₓ, ketoconazole decreased in a biphasic manner for up to 12 hours (last time point for sample collection).

The mean (SD) plasma AUC(0-12) for ketoconazole on Day-1 was 35260 (16917) ng*h/mL. On Day 1 following ketoconazole administration, the median Tₘₐₓ was also reached at 2 hours. The Day 1 mean (SD) Cₘₐₓ achieved was 6615 (1589.6) ng/mL. The mean (SD) CL/F of ketoconazole was estimated to be 7.954 (7.110) and 5.717 (2.373) L/h, respectively, on Day-1 and Day 1.

There was 21-22% increased exposure of ketoconazole when given with betrixaban (Table 3).

Based on a linear mixed model with scheduled time as a fixed and repeated effect to the log-transformed trough concentrations of ketoconazole, attainment of steady state could not be demonstrated. The least squares geometric mean estimates for the ketoconazole trough concentrations on Day-1 predose, Day-1 12 hours, Day 1 predose, and Day 1 12 hours were 1250, 697, 1030, and 769 ng/mL respectively.

TABLE 3
Effect of co-administration of betrixaban on key
ketoconazole plasma pharmacokinetic parameters

| Parameter | | | Geometric | Pairwise Comparisons | | | |
|---|---|---|---|---|---|---|---|
| (unit) | Treatment | n | LS Mean | Pair | Ratio (%) | 90% CI | p-value |
| Cₘₐₓ | R | 12 | 5265 | T/R | 121.9 | (100.3, 148.1) | 0.0952 |
| (ng/mL) | T | 12 | 6417 | | | | |
| AUC₍₀₋₁₂₎ | R | 12 | 30910 | T/R | 120.8 | (98.8, 147.7) | 0.1198 |
| (ng*h/mL) | T | 12 | 37330 | | | | |

Notes:
Treatment T: ketoconazole 200 mg co-administered with 40-mg betrixaban; Treatment R: ketoconazole 200 mg alone.
LS = Least squares;
CI = Confidence interval.
Based on fitting a linear mixed model with treatment as a fixed effect and subject as a random effect to the log-transformed values.

In the present study, pharmacokinetics of betrixaban were evaluated in healthy subjects after 40 mg oral administration of betrixaban either alone or following 5 days of treatment with ketoconazole (200 mg every 12 hours). The study aimed at evaluating the effects of ketoconazole (a P-gp inhibitor) on the PK of betrixaban. betrixaban reached a median maximal plasma concentration at 1 hour in both the betrixaban alone and betrixaban+ketoconazole groups. This median Tₘₐₓ was similar to the median Tₘₐₓ observed in previous studies where healthy subjects were dosed with single oral doses of betrixaban.

The exposure [Cₘₐₓ, AUC_{(0-Tlast)} and AUC_{(0-∞)}] of betrixaban was approximately 2 fold higher in the group treated with betrixaban with ketoconazole as compared to betrixaban alone. Since this was a randomized crossover design, the same subjects were used for both the treatment. All of the 11 subjects who completed both the treatments showed an increase in the betrixaban AUC_{(0-∞)} and AUC_{(0-Tlast)} when given with ketoconazole. Ten out of the 11 subjects showed an increase in betrixaban Cₘₐₓ when given with ketoconazole. The CIs for the geometric least-squares mean ratios for the treatments were completely outside the 80 to 125% limits for AUC_{(0-∞)}, AUC_{(0-Tlast)} and Cₘₐₓ Thus ketoconazole significantly affected the PK of betrixaban after oral administration.

Differences were also seen in the two groups with regards to the terminal elimination half-life of betrixaban with the terminal elimination half-life being slightly higher in the betrixaban alone group as compared to the betrixaban+ketoconazole group. Similar to the terminal elimination half-life the oral clearance (CL/F) and the volume of distribution (Vz/F) were higher for the betrixaban alone group as compared to the betrixaban+ketoconazole group.

Ketoconazole is an inhibitor of Pgp. In addition, ketoconazole is also an inhibitor of CYP3A. It was also observed that betrixaban is not significantly metabolized by the CYP isoenzymes. The increase in the exposure of betrixaban when administered with ketoconazole is therefore most likely due to the inhibition of Pgp and not CYP3A. Pgp is expressed in the gastrointestinal tract as well as in the renal tubule and the biliary tract. Thus the inhibition of Pgp, which can have significant effects on the drug exposure, can occur at any of these Pgp expression sites. Considering that there were significant differences in the terminal elimination half-life of betrixaban when administered with ketoconazole, it is possible that not just the absorption but even the elimination of betrixaban is affected by ketoconazole.

Betrixaban was eliminated unchanged in the urine to an extent of 2.8% without ketoconazole and 6.6% with ketoconazole in 72 hours. Applicant has also observed that that the urinary excretion is not a major route for the elimination of betrixaban. In addition, no differences were seen in the estimated renal clearance of betrixaban between the betrixaban alone and betrixaban+ketoconazole groups. It is therefore unlikely that changes in urinary excretion can explain the observed differences in the PK exposure of betrixaban.

The ketoconazole PK was also examined in this study on Day-1 and Day 1, with the Day 1 being the day of treatment with betrixaban. There was no significant difference observed in the ketoconazole PK on the 2 different days though the levels on Day 1 (when ketoconazole was given with betrixaban) were slightly higher. Thus inhibition of Pgp by ketoconazole or co-administration of betrixaban with ketoconazole does not significantly change the PK of ketoconazole.

In sum, this example demonstrates that ketoconazole significantly influences the pharmacokinetics of betrixaban after oral administration. There is an approximately 2.1 fold increase in the plasma AUC_{(0-∞)} and 2.3 fold increase in the plasma Cₘₐₓ of betrixaban when administered with ketoconazole as compared to when administered alone. Further, ketoconazole administration does not appear to have an effect on the renal clearance of betrixaban.

When co-administered with betrixaban, there is ˜20% increased exposure of ketoconazole [Cₘₐₓ and AUC₍₀₋₁₂₎]. Finally, oral administration of a single-dose betrixaban 40-mg capsule alone and following 5 days of ketoconazole was well tolerated in this study.

### Example 3

### Verapamil Increases the Exposure of Betrixaban

Example 1 shows that amiodarone use results in an approximately 2.5-2.7 fold increase in betrixaban C₁₂ₕᵣ. Example 2, likewise, revealed a 2.2-fold increase in AUC and a 2.4-fold increase in Cₘₐₓ for betrixaban with ketoconazole compared to administration alone. Verapamil is a 2-4 fold less potent Pgp inhibitor than ketoconazole (based on in vitro assays). This example, however, discovered unexpectedly that co-administration of verapamil increased the exposure of betrixaban to an even greater extent.

### Methods

This example uses a clinical trial that was an open-label, 2-period, fixed-sequence study to evaluate the influence of single and multiple oral doses of verapamil on the single-dose pharmacokinetics of betrixaban. About twenty (20) healthy male or female subjects received 2 different treatments, Treatment A in Period 1 and Treatment B in Period 2 in a fixed sequence design. Period 1 (Treatment A) consisted of a single dose of 40 mg betrixaban. Period 2 (Treatment B) consisted of 240 mg of verapamil HCl SR QD (2 of 120 mg verapamil tablets) for 18 days with single doses of 40 mg betrixaban co-administered with verapamil on Days 1 and 14. All study drug was administered in the fasted state after an overnight fast with 240 mL of water, with water restricted 1 hour prior and 1 hour after study drug administration. Period 2 was no sooner than 10 days after betrixaban dosing in period 1. Blood samples for betrixaban assay were collected at selected time points for up to 120 hours postdose for determination of betrixaban pharmacokinetic profile in the presence and absence of verapamil.

### Results

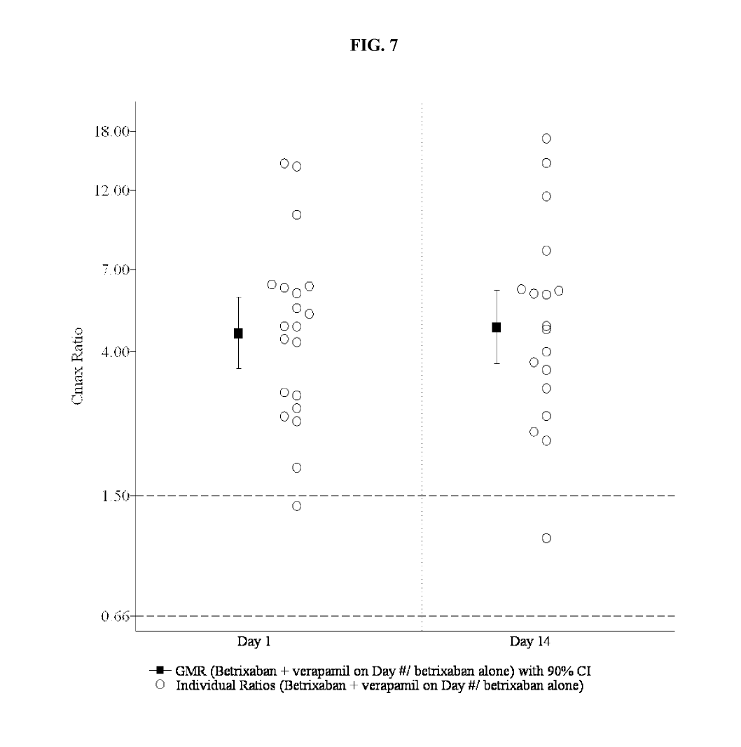

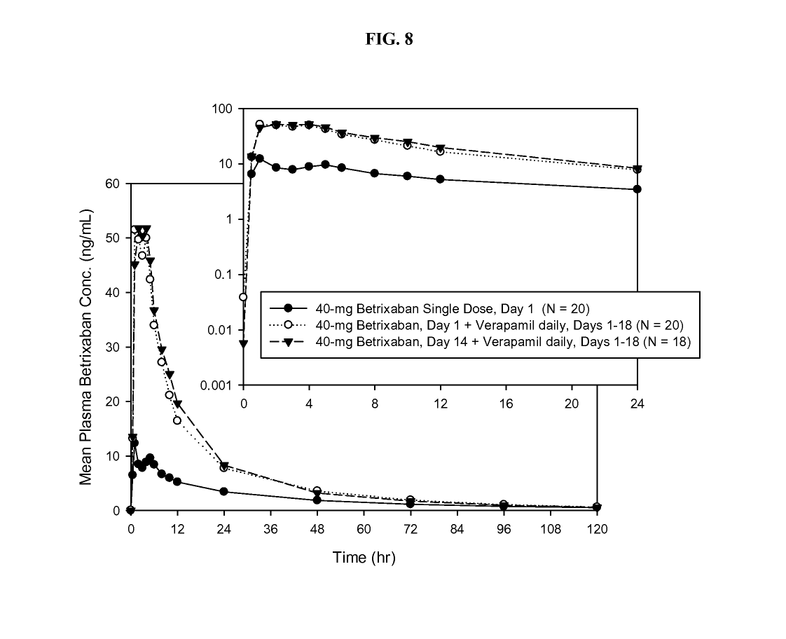

Table 4 listed summary statistics and statistical comparisons for the plasma PK parameters of betrixaban after co-administration with verapamil on Days 1 and 14 in healthy volunteers. Individual and geometric mean ratios of AUC_{0-∞} and Cₘₐₓ were depicted in FIGS. 6 and 7, respectively. Mean plasma concentration profiles for betrixaban at all treatments were shown in FIG. 8.

TABLE 4
Summary statistics and statistical comparisons for the plasma pharmacokinetic
parameters of betrixaban after co-administration with verapamil on days 1 and 14 in
healthy volunteers

| | | | | | Apparent |
|---|---|---|---|---|---|
| Treatment | N | AUC_{0-∞}^{†} (hr * ng/mL) | Cₘₐₓ^{†} (ng/mL) | Tₘₐₓ^{‡} (hr) | t_{1/2}^{§} (hr) |
| Betrixaban alone | 20 | 264.20 (218.93, 318.81) | 13.09 (10.13, 16.93) | 1.0 (0.5, 8.0) | 40.2 (7.0) |
| Betrixaban + verapamil | 20 | 762.65 (631.99, 920.32) | 59.63 (46.12, 77.09) | 2.0 (1.0, 5.0) | 27.4 (4.1) |
| on Day 1 | | | | | |
| Betrixaban + verapamil | 18 | 802.12 (660.84, 973.61) | 62.07 (47.47, 81.16) | 2.5 (0.5, 5.0) | 29.3 (5.8) |
| on Day 14 | | | | | |

| Comparison | AUC_{0-∞}^{\|\|} | Cₘₐₓ^{\|\|} |
|---|---|---|
| Betrixaban + verapamil on Day 1/betrixaban alone | 2.89 (2.49, 3.34) | 4.55 (3.57, 5.80) |
| Betrixaban + verapamil on Day 14/betrixaban alone | 3.04 (2.61, 3.54) | 4.74 (3.69, 6.09) |

^{†}Geometric mean back-transformed from log scale (95% CI).
^{‡}Median (Minimum, Maximum).
^{§}Harmonic mean (Jackknife SD).
^{||}GMR (90% CI).
^{¶}rMSE for AUC_{0-∞} = 0.275 and rMSE for Cₘₐₓ = 0.454; rMSE: Square root of conditional mean squared error (residual error) from the linear mixed effect model. rMSE * 100% approximates the within-subject % CV on the raw scale.
GMR = Geometric least-squares mean ratio between treatments;
CI = Confidence interval.

Preliminary PK results suggest single-dose betrixaban AUC_{0-∞} and Cₘₐₓ were increased by ˜3- and 4.5˜ fold when coadministered with both single-dose and multiple-dose verapamil compared to being administered alone. The Day 1 AUC_{0-∞} and Cₘₐₓ geometric least-squares mean ratio (GMRs) (90% CIs) for [betrixaban+verapamil/betrixaban alone] were 2.89 (2.49, 3.34) and 4.55 (3.57, 5.80), respectively. The Day 14 AUC_{0-∞} and Cₘₐₓ GMRs (90% CIs) for [betrixaban+verapamil/betrixaban alone] were 3.04 (2.61, 3.54) and 4.74 (3.69, 6.09), respectively. The 90% CIs for GMRs of AUC_{0-∞} and Cₘₐₓ were not contained within the (0.66, 1.50) target interval on both Days 1 and 14, not supporting the hypothesis that single or multiple oral dose administration of verapamil does not substantially influence the AUC_{0-∞} or Cₘₐₓ of a single 40-mg oral dose of betrixaban. The GMRs (Day 14/Day 1) of AUC_{0-∞} and Cₘₐₓ were 1.05 and 1.04, respectively, which indicated that no additional inhibition/induction occurred between single dose of verapamil and steady-state.

The effect of verapamil on betrixaban, particularly on Cₘₐₓ, was variable. Although the betrixaban geometric mean Cₘₐₓ was about 60 ng/mL with verapamil on average on both Days 1 and 14, several subjects had Cₘₐₓ values in excess of 100 ng/mL (the highest mean Cₘₐₓ tested in the tQT study). Overall, pharmacokinetic variability was fairly high both with and without verapamil; the AUC_{0-∞} and Cₘₐₓ % CVs were ˜60 and 88%, respectively (betrixaban alone); ˜39 and 64%, respectively, on Day 1 and ˜34 and 41%, respectively, on Day 14 (betrixaban with verapamil). Given that the absolute bioavailability of betrixaban in the fasted state is about 32%, the observed effect of verapamil on AUC (˜3-fold increase) suggests that close to maximal exposure may have been achieved and that Pgp-mediated drug efflux markedly limits oral bioavailability.

Betrixaban concentration-time profiles are characterized by dual absorption peaks. Inspection of individual profiles suggests that the incidence of dual peaks tend to diminish as the first peak becomes more prominent for betrixaban with verapamil compared to betrixaban alone. No substantial differences in Tₘₐₓ were observed between treatments. The apparent terminal t_{1/2} was shorter for betrixaban with verapamil (˜30 hr) compared to alone (˜40 hr). The slight differences in terminal t_{1/2} may be due to Pgp induction due to verapamil, which was observed to occur very rapidly (within 3 hrs) in vitro, although other studies have shown no inductive potential for verapamil.

In sum, single-dose betrixaban AUC_{0-∞} and Cₘₐₓ were increased by ˜3- and 4.5˜ fold when co-administered with both single-dose and multiple-dose verapamil compared to being administered alone (Table 5). The 90% CIs for GMRs of AUC_{0-∞} and Cₘₐₓ were not contained within the (0.66, 1.50) target interval on both Days 1 and 14, not supporting the hypothesis that single or multiple oral dose administration of verapamil does not substantially influence the AUC_{0-∞} or Cₘₐₓ of a single 40-mg oral dose of betrixaban. The observed GMRs (Day 14/Day 1) of AUC_{0-∞} and Cₘₐₓ were 1.05 and 1.04, respectively, which indicated that no additional inhibition/induction occurred between single dose of verapamil and steady-state, and that inhibition of Pgp apparently outweighed any possible inductive effects at steady-state.

TABLE 5
Geometric least-squares mean ratio (GMR) of verapamil's effect
on exposure of betrixaban

| | GMR (90% CI) | |
|---|---|---|
| Comparison (n = 20) | AUC_{0-∞} | Cₘₐₓ |
| betrixaban + verapamil on Day 1/ | 2.89 (2.49, 3.34) | 4.55 (3.57, 5.80) |
| betrixaban alone | | |
| betrixaban + verapamil on Day 14/ | 3.04 (2.61, 3.54) | 4.74 (3.69, 6.09) |
| betrixaban alone | | |

The elevations in betrixaban concentrations with verapamil were higher than expected based on the prior results for coadministration with potent Pgp inhibitors. The results reveal that moderate Pgp inhibitors may have larger than anticipated effects on Cₘₐₓ, and that in vitro potency of Pgp inhibition may not enable adequate prediction of potential with betrixaban. The study in Example 2 with ketoconazole revealed a 2.2-fold increase in AUC_{0-∞} and a 2.4-fold increase in Cₘₐₓ for betrixaban with ketoconazole compared to administration alone. Likewise, Example 1 suggested that amiodarone use resulted in approximately a 2.5-2.7 fold increase in betrixaban C₁₂ₕᵣ.

It is noted that in the current study, betrixaban and verapamil were co-administered concurrently, while in Example 2 betrixaban was administered 1 hour after ketoconazole. In Example 1, betrixaban was dosed 2 hours after the evening meal. Of 42 patients in this study, 3 reported having taken amiodarone at bedtime, while the others reported a.m. dosing of amiodarone. Thus, time of dosing a Pgp inhibitor relative to betrixaban administration likely contributes to the magnitude of the effect (Table 6).

TABLE 6
Comparison of P-glycoprotein inhibitors on their effect on exposure of betrixaban

| | | | Point estimate of GMR | |
|---|---|---|---|---|
| | | | [betrixaban + verapamil/ | |
| Pgp inhibitor | Betrixaban | | betrixaban alone] | |
| (Regimen) | Dose | Timing | AUC_{0-∞} | Cₘₐₓ |
| Ketoconazole | Single | Keto 1 h prior to | 2.2 | 2.4 |
| 200 mg | 40 mg, fasted | Betrixaban | | |
| (BID × 5 days) | | | | |
| Verapamil SR | Single | Concurrent | 2.9 (Day 1) | 4.5 (Day 1) |
| 240 mg | 40 mg, fasted | Administration | 3.0 (Day 14) | 4.7 (Day 14) |
| (QD × 14 days) | | | | |

| | | | |
|---|---|---|---|
| Amiodarone | 40-80 mg QD | Betrixiban dosed 2 hr | C12 hr increased 2.5-2.7-fold |
| (Individualized | at steady state, | after evening meal; | (from a POP PK analysis using data |
| dose/regimen) | fed | amiodarone usually | from EXPLORE Xa Ph IIb) |
| | | dosed in the morning | |

These data suggest that a relatively weaker Pgp inhibitor could have a more marked effect on betrixaban if apical intestinal concentrations are substantial (in individual cases not clear whether intestinal lumen or plasma levels or both are primary determinants of Pgp inhibition).

In addition, the permeability and/or solubility of the inhibitor may have a large impact on the magnitude of the effect in the intestine (Collett et al., “Rapid induction on P-glycoprotein expression by high permeability compounds in colonic cells in vitro: a possible source of transporter mediated drug interactions,” Biochemical Pharmacology 2004; 68: 783-790.), and thus the effect on first pass drug efflux. It should be noted that verapamil (BCS class I), ketoconazole (BCS class II), and amiodarone (BCS class II), are all high permeability compounds, while verapamil is also high solubility. Finally, the specific PK profile of the inhibitor may impact the DDI results. In this case an extended release formulation of verapamil (verapamil SR) was used. An immediate release formulation of verapamil had a more pronounced effect on dabigatran (a direct thrombin inhibitor and Pgp substrate), compared to verapamil SR (Dabigatran Advisory Committee Briefing Document, 27 Aug. 2010, Sec. 4.4.).

In conclusion, the timing of administration of a Pgp inhibitor in relation to betrixaban dosing likely has an impact on betrixaban elevations. Other factors, such as the PK profile of the inhibitor (immediate or extended release), administration fasted or with food, as well as GI transit times, could have profound effects on the net result. Finally, the potential for substantial increases in mean and individual Cₘₐₓ values are to be evaluated in light of the potential for QT interval prolongation at high betrixaban concentrations.

### Example 4

### Co-Administration with Digoxin does not Change the Exposure of Betrixaban

Despite the findings in Examples 1-3 that co-administration with a Pgp inhibitor, e.g., amiodarone, ketoconazole, or verapamil, increases the exposure of betrixaban, the current example demonstrates that the same synergistic effect does not exist for digoxin, another Pgp inhibitor.

### Methods

In this single-center, open-label, sequence-randomized, 3-period crossover study of betrixaban and digoxin, each drug was administered alone and in combination for 7 days to 18 healthy subjects. The dose of betrixaban was 80 mg once daily. A loading dose of digoxin (0.75 mg total) was given on Day 1 followed by a maintenance dose of 0.25 mg/day. For each period, subjects reported to the clinical research unit on the day prior to Day 1 and remained there until Day 8 (at least until the last blood sample collection). The total duration of the study for each subject was 14 weeks: up to 4 weeks predose, approximately 1 week in the study unit on 3 occasions (with each treatment period separated by an approximate 2-week washout [i.e., 12 to 14 days]), and up to 3 weeks after the last dose until the Termination Visit. Serial blood samples and interval urine collections were obtained over the last dosing interval (Day 7 to 8) during each study period. Routine safety laboratory data was obtained at baseline during and after drug administration; additional safety laboratory and clinical data were collected.

A total of 18 subjects were enrolled in the study, and 14 subjects completed the study. All 18 subjects were included in the PK and safety analyses.

Subjects were randomized to receive daily oral doses of betrixaban and digoxin in combination (Test Treatment C) for 7 days. All doses were taken under fasting conditions (no food starting from the midnight before dosing and continuing until 2 hours postdose).

Betrixaban maleate (betrixaban) 40 mg capsules were provided by Portola Pharmaceuticals, Inc. Lanoxin® (Digoxin) 0.25 mg tablets were manufactured by GlaxoSmithKline. Subjects who received Treatment A were administered a once-daily oral dose of 2 betrixaban maleate (betrixaban) 40 mg capsules with 240 mL of water for 7 days.

On Day 1, subjects who received Treatment B were administered a single oral dose of 2 Lanoxin® (Digoxin) 0.25 mg tablets with 240 mL of water followed by a single oral dose of 1 Lanoxin® (Digoxin) 0.25 mg tablet with 240 mL of water 6 hours later. Then on Days 2-7, subjects were administered a once-daily oral dose of 1 Lanoxin® (Digoxin) 0.25 mg tablet with 240 mL of water.

On Day 1, subjects who received Treatment C were administered a single oral dose of 2 betrixaban maleate (betrixaban) 40 mg capsules and 2 Lanoxin® (Digoxin) 0.25 mg tablets with 240 mL of water followed by a single oral dose of 1 Lanoxin® (Digoxin) 0.25 mg tablet with 240 mL of water 6 hours later. On Days 2-7, subjects who received Treatment C were administered a single oral dose of 2 betrixaban maleate (betrixaban) 40 mg capsules and 1 Lanoxin® (Digoxin) 0.25 mg tablet with 240 mL of water once daily.

Subjects were randomized to receive daily oral doses of betrixaban or digoxin, each drug administered alone (Reference Treatments A and B, respectively).

Blood samples for determination of plasma levels of betrixaban and digoxin were obtained at the following time points: just prior to dosing (Hour 0) on Days 1 through 7, and on Day 7 at 1, 1.5, 2, 2.5, 3, 3.5, 4, 4.5, 5, 5.5, 6, 7, 8, 10, 12, 15, and 24 hours after the last dose. Urine samples for betrixaban and digoxin determination was obtained prior to the first dose on Day 1 (Hour 0) and from 0-12 and 12-24 hours post last dose on Day 7.

Safety assessments included electrocardiogram (ECG) intervals, vital signs, laboratory parameters, and adverse events (AEs).

Plasma betrixaban, its metabolites, and digoxin PK parameters included Cₘₐₓ, Cₘᵢₙ, tₘₐₓ, and AUC0-24. In addition, % AUC0-24 was calculated for the betrixaban metabolites. All concentration and PK results were summarized using appropriate descriptive statistics (mean, standard deviation [SD], coefficient of variation [CV %], minimum, maximum, median, and geometric mean). Mean and individual concentration-versus-time curves were plotted. Urine betrixaban, its metabolites, and digoxin PK parameters included amount excreted (Ae), cumulative amount excreted (Ae0-24), renal clearance (CLr), and % dose excreted. Individual PK parameters in urine were listed and summarized with descriptive statistics (mean, SD, CV %, minimum, maximum, and median).

Attainment of steady state for betrixaban, its metabolites, and digoxin was assessed by linear regression of the trough concentrations versus day (Day 5, 6, and 7). Steady state was concluded if the slope was not significantly different from zero (p>0.05). A parametric (normal-theory) mixed model was applied to the ln-transformed Cₘₐₓ and AUC₀₋₂₄ values on Day 7 for betrixaban and digoxin. The 90% confidence intervals (CIs) for the difference in least squares means (test-reference, where test=drugs in combination and reference=drugs alone) was calculated for each parameter. The resulting confidence limits were exponentiated and reported on the original measurement scale. Lack of clinically significant drug interaction was concluded if the 90% CIs were within the range of 80% to 125%.

### Results

Co-administration of daily oral doses of digoxin and betrixaban for 7 days resulted in minimal changes in the PK parameters of betrixaban when compared to betrixaban administered alone. The arithmetic means for plasma betrixaban Cₘₐₓ and AUC0-24 were similar between the 2 treatments (92.5 ng/mL versus 92.6 ng/mL and 943.9 versus 935.5 ng*hr/mL, respectively).

Urine PK parameters (Cum. Ae, CLr, and Cum. % dose excreted) were also similar between the 2 treatments. Median tₘₐₓ was approximately 1 hour earlier following co-administration of digoxin compared to betrixaban administered alone (2.52 versus 3.50 hours). However, tₘₐₓ range values were comparable between both treatments (0.994-4.50 versus 1.00-4.55 hours).

The 90% CI for ln-transformed AUC0-24 fell within the 80% to 125% range, indicating digoxin had no effect on the betrixaban exposure following repeated daily oral doses for 7 days. However, for ln-transformed Cₘₐₓ the lower limit of the 90% CI (75.6%) fell below the 80% lower boundary of the acceptable 80 to 125% range, and consequently, absence of drug interaction of digoxin on betrixaban Cₘₐₓ could not be formally concluded. The 90% CI encompassed the value of 100%, indicating that the small difference may not be statistically or clinically relevant.

After daily dosing for 7 days of betrixaban capsules alone and in combination with digoxin tablets, betrixaban appeared to have reached steady state by Day 6, as shown by visual assessment of trough values. However, steady-state p-values were 0.0016 and 0.0134, respectively, indicating that steady-state conditions may not have been established by Day 7 for either treatment or the variability was too high, confounding the analysis.

Co-administration of daily oral dose of betrixaban and digoxin for 7 days resulted in minor changes in the PK parameters of digoxin when compared to digoxin administered alone. The arithmetic means for plasma betrixaban Cₘₐₓ and AUC0-24 were similar between the 2 treatments (1.76 versus 1.61 ng/mL and 16.2 versus 15.3 ng*hr/mL, respectively). Urine PK parameters (Cum. Ae, CLr, and Cum. % dose excreted) were also similar between the 2 treatments.

Median tₘₐₓ of digoxin remained constant after both treatments (1.00 versus 1.01 hours). In addition, the 90% CIs for ln-transformed Cₘₐₓ and AUC0-24 fell within the 80% to 125% range, indicating that co-administration of betrixaban had no impact on digoxin PK. After daily doses for 7 days of digoxin tablets alone and in combination with betrixaban capsules, steady-state concentrations appeared to have been reached by Day 5, as shown by visual assessment of trough values. However, steady-state p-values were 0.0037 and 0.0073, respectively, indicating that steady-state conditions may not have been established by Day 7 for either treatment or the variability was too high, confounding the analysis.

In sum, the co-administration of betrixaban and digoxin over 7 days had no effect on the PK of digoxin or the AUC of betrixaban.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. A method for treating thrombosis or inhibiting blood coagulation in a patient receiving administration of a P-glycoprotein inhibitor, the method comprising administering to the patient a subtherapeutic dose of betrixaban.

2. The method of claim 1, wherein the amount of betrixaban administered is about 20% less than the therapeutically effective amount.

3. The method of claim 1, wherein the amount of betrixaban administered is about 50% less than the therapeutically effective amount.

4. The method of claim 1, wherein the patient is a human patient and the patient is administered an aggregate daily dose of about 25 to about 35 mg of betrixaban.

5. The method of claim 1, wherein the patient is a human patient and the patient is administered an aggregate daily dose of about 10 to about 20 mg of betrixaban.

6. (canceled)

7. The method of claim 1, wherein the patient receives the administration of the P-glycoprotein inhibitor at least half an hour before or after administration of betrixaban.

8. The method of claim 1, wherein the patient is concurrently administered with the P-glycoprotein inhibitor and betrixaban.

9. The method of claim 1, wherein the patient receives administration of an therapeutically effective amount of the P-glycoprotein inhibitor.

10. The method of claim 1, wherein the P-glycoprotein inhibitor is in a controlled release form.

11. The method of claim 1, wherein the P-glycoprotein inhibitor is selected from the group consisting of verapamil, amiodarone and ketoconazole.

12. The method of claim 11, wherein the P-glycoprotein inhibitor is verapamil.

13. The method of claim 12, wherein verapamil is administered in an amount of about 100 mg to about 300 mg.

14. The method of claim 11, wherein the P-glycoprotein inhibitor is amiodarone.

15. The method of claim 14, wherein amiodarone is administered in an amount of about 200 mg to about 400 mg.

16. The method of claim 11, wherein the P-glycoprotein inhibitor is ketoconazole.

17. The method of claim 16, wherein ketoconazole is administered in an amount of about 100 mg to about 300 mg.

18. (canceled)

19. The method of claim 1, wherein the betrixaban is a maleate salt.

20. The method of claim 19, wherein the maleate salt is in a crystalline form selected from the group consisting of Form I, Form II, Form III and combinations thereof.

21-24. (canceled)

25. The method of claim 1, wherein the patient is a patient with atrial fibrillation or atrial flutter.

26. An unit dose comprising from about 25 to about 35 mg of betrixaban and an effective amount of a P-glycoprotein inhibitor.

27-28. (canceled)
